(19) United States
(12) Patent Application Publication
(10) Pub. No.: US 2009/0069196 A1
(43) Pub. Date: Mar. 12, 2009

(54) Prediction of Breast Cancer Response to Chemotherapy

(76) Inventors: Mathias Gehrmann, Leverkusen (DE); Christian Von Toerne, Solingen (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/281,780

(22) PCT Filed: Mar. 9, 2007
(86) PCT No.: PCT/EP07/02063

§ 371 Date: Sep. 5, 2008

(30) Foreign Application Priority Data
Mar. 22, 2006 (EP) 06005837.7

(51) Int. Cl. C40B 40/06; C12Q 1/68; C40B 60/12

(52) U.S. Cl. 506/16; 435/6; 506/39

(57) ABSTRACT

Method for the prediction of the response to epirubicin/cyclophosphamide-based chemotherapy of a breast cancer in a patient, from a tumour sample of said patient, comprising steps of determining the expression level of a group of marker genes consisting of (i) a first marker gene selected from the group consisting of MLPH, SPDEF, and AKR7A3; and (ii) a pair of second marker genes selected from the group of pairs consisting of (H2BFS and UBE2S), (BGN and ZBTB16), (ZBTB16 and EMP1), (LGALS8 and UBE2S) and (OLFML2B and ZBTB16); and (iii) a third marker gene selected from the group consisting of CYBA, ACP5, a gene specifically binding to Affymetrix probe set ID 210915 x at, LCK, GSTM3; classifying said sample as belonging to one of several breast cancer response classes from the expression levels determined; predicting the response of said breast cancer in said patient to chemotherapy from previously known characteristic properties of tumours of said one of several breast cancer response classes.

### TECHNICAL FIELD OF THE INVENTION

The present invention relates to methods and kits for the prediction of a likely outcome of chemotherapy in a cancer patient. More specifically, the invention relates to the prediction of tumour response to chemotherapy based on measurements of expression levels of a small set of marker genes. The set of marker genes is useful for the identification of breast cancer subtypes responsive to e.g. epirubicin/cyclophosphamide (EC) based chemotherapy.

### BACKGROUND OF THE INVENTION

Breast cancer is one of the leading causes of cancer death in women in western countries. More specifically breast cancer claims the lives of approximately 40,000 women and is diagnosed in approximately 200,000 women annually in the United States alone. Over the last few decades, adjuvant systemic therapy has led to markedly improved survival in early breast cancer (EBCTCG, 1998 a+b). This clinical experience has led to consensus recommendations offering adjuvant systemic therapy for the vast majority of breast cancer patients (Goldhirsch et al., 2003). In breast cancer a multitude of treatment options are available which can be applied in addition to the routinely performed surgical removal of the tumour and subsequent radiation of the tumour bed.

Chemotherapy may be applied postoperative, i.e. in the adjuvant setting or preoperative, that is in the neoadjuvant setting in which patients receive several cycles of drug treatment over a limited period of time, before remaining tumour cells are removed by surgery. In the past, neoadjuvant chemotherapy had been used for patients with locally advanced breast cancer. More recently, patients with large tumours become treated with neoadjuvant therapy as well. Primary goal is a reduction of tumour size in order to increase the possibility of breast-conserving treatment.

Yet, most if not all available drug treatments have numerous adverse effects which can severely impair patients' quality of life (Shapiro and Recht, 2001; Ganz et al., 2002). This makes it mandatory to select the treatment strategy on the basis of a careful risk assessment for the individual patient to avoid over- as well as under treatment. Hence, it is desirable to have available a methods for the prediction of the response of a patient to a particular chemotherapy prior to the actual onset of said chemotherapy. This allows for the best possible chemotherapeutic regimen to be selected for a particular patient.

Folgueira et al. (2005, Clin. Cancer Res., 11(20), pp. 7434-7443) disclose a method for the prediction of the response of cancer patients to doxorubicin-based primary chemotherapy. Patients were classified in two groups, namely responders and non-responders. The classification is based on a trio of marker genes (PRSS11, MTSS1, CLPTM1) which correctly distinguished 95.4% of 44 samples analysed, with only two misclassifications. The classification is a single step classification. Folgueira et al., however, do not disclose marker genes or methods for the prediction of the response to epirubicin/cyclophosphamide (EC) based chemotherapy.

Ayers et al (2004, J. Clin. Oncology, 11(12), pp. 2284-2293) examine the feasibility of developing a multigene predictor of pathologic complete response to sequential weekly paclitaxel and fluorouracil+doxorubicin+cyclophosphamide (T/FAC) neoadjuvant chemotherapy for breast cancer. A multi-gene model with 74 marker genes was built. The authors conclude that transcriptional profiling has the potential to identify a gene expression pattern in breast cancer that may lead to clinically useful predictors of pathological complete response to T/FAC neoadjuvant therapy. The authors, however, do not disclose marker genes for the response prediction in EC-based neoadjuvant chemotherapy.

Hannemann et al. (2005, J. Clin. Oncology, 23(15), pp. 3331-3342) investigated whether clinically useful markers predicting response of primary breast carcinomas to either doxorubicin-cyclophosphamide (AC) or doxorubicin-docetaxel (AD) could be identified. Patients were classified into three breast cancer response classes (pathologic complete response, partial remission, no response). However, no gene expression profile predicting the response of primary breast carcinomas to AC- or AD-based chemotherapy could be found in this study. This study furthermore did not attempt to identify a method for the prediction of the response to EC-based neoadjuvant chemotherapy.

Rouzier et al. (2005, Clin. Cancer Res., 11(16), pp. 5678-5685) disclose a molecular classification of breast cancer into “luminal”, “basal-like”, “normal-like” and erbB2+” subgroups. These subgroups show different rates of pathologic complete response to 5-fluorouracil, doxorubicin and cyclophosphamide neoadjuvant chemotherapy. The classification algorithm applies 424 genes to separate the four groups in a single step classification scheme. This study, however, does not provide a method to predict the response to EC-based neoadjuvant chemotherapy.

Van't Veer et al. (2002, Nature, 415, pp. 530-536) disclose a method for the prognosis of the disease outcome in breast cancer patients on the basis of gene expression profiling experiments. A set of “prognosis reporter genes” was identified which separates patients with “good” (no distant metastases within 5 years) and “bad prognosis” (distant metastases within 5 years). Van't Veer et al., however, do not provide a method for the response prediction to chemotherapy, in particular not to EC-based chemotherapy.

Wang et al. (2005, Lancet, 365, pp. 671-679) identified patterns of gene activity that subclassify tumours to provide means for individual risk assessment in patients with lymph-node negative breast cancer. These gene signatures allow for the identification of patients at high risk of distant recurrence in a multi-step identification procedure. This publication relates to the prognosis of breast cancer outcome only, but not to methods for the prediction of response to chemotherapy, in particular not to EC-based chemotherapy.

WO 04/111603, assigned to Genomic Health Inc., discloses sets of genes the expression of which is useful for predicting whether cancer patients are likely to have beneficial treatment response to chemotherapy. Numerous marker genes are identified and used, alone or in combination with other marker genes, to predict breast cancer response. WO 04/111603, however, does not disclose a method for the prediction of the response of a breast cancer patient to EC-based neoadjuvant chemotherapy.

Modlich et al. (2005, Journal of Translational Medicine 3(32), http://www.translational-medicine.com/content/3/1/32) disclose a method for the prediction of the response of breast cancer tumours to EC-based chemotherapy. Breast cancer patients were classified into three classes (pathologically confirmed complete remission, partial remission, no change) in a classification scheme following a decision tree. A “favourable outcome” gene signature consisting of 31 genes was identified, which separates complete responders from the remaining classes, i.e. partial responders and no change patients (“poor outcome group”). A “poor outcome signature” consisting of 26 marker genes was identified which allows separation of partial responders and no change patients in the “poor outcome group”. The disclosed method, however, uses a large number of marker genes to separate breast cancer response classes, said marker genes being different from the ones used according to the present invention. Using a large number of marker genes (as opposed to only a few highly informative marker genes) makes both the experiments and the statistical analysis more difficult to perform. The method of the invention, uses a low number of highly informative marker genes, and separates breast cancer patients into breast cancer response classes in a simple but highly accurate manner. Separation into four distinct breast cancer response classes, as provided by the present invention, also allows for a more detailed prediction of patient response.

Accurate prediction of the response of a breast cancer patient to EC-based chemotherapy could help to select the most efficient and appropriate drug for breast cancer treatment in the patient, providing a means of individualized patient care. Thus, there is a need in the art for reliable methods of predicting the response of breast cancer patients to EC-based neoadjuvant chemotherapy.

### SUMMARY OF THE INVENTION

The present invention is based on the unexpected finding that robust classification of breast tumour tissue samples into clinically relevant subgroups can be achieved by classifiers that use a small set of expression values of specific marker genes. The subgroups, as defined by the classification algorithm of the invention, represent EC response classes which are characterized by a particular likelihood of tumour response to neoadjuvant EC-based chemotherapy. Using the expression values of the small set of marker genes a plurality of algorithms can be employed to perform the task of robust classification of an unknown sample into one of the response classes. Preferably, the EC response class of a tumour is predicted hierarchically by separating a number of mutually disjoint aggregate or elementary classes at a time (cf. FIG. 1), i.e. by using a “classification tree”. In each node of this tree a partial classification is performed on the basis of a very small number of genes. Preferably, each separation step in the classification tree is achieved on the basis of the expression of a single specific marker gene, or a single pair of specific marker genes. Each single marker gene can be substituted by further marker genes, provided the expression values of the further marker gene exhibit a high degree of correlation to the RNA expression values of the marker gene. These genes are used to reliably distinguish aggregate and elementary classes until the sample can uniquely be assigned to its elementary class (the leaves of the tree structure).

Sets of marker genes are provided for the classification of a breast tumour into one of several breast cancer response classes. These sets of marker genes can be used to predict a patient's response to EC-based chemotherapy.

Hence the current invention provides means to decide—shortly after tumour biopsy—whether or not a certain mode of chemotherapy is likely to be beneficial to the patient's health and/or whether to maintain or change the applied mode of chemotherapy treatment.

Kits and devices for performing the above methods are further aspects of the invention.

### BRIEF DESCRIPTION OF THE FIGURES

FIG. 1: Decision tree for classification of breast cancer tissues into EC response classes A, B, C, and D, based on marker gene expression measurements.

FIG. 2: Hypothetical data set with Gene X and Gene Y, and 2 distinct classes, 500 samples per class.

FIG. 3: Histogram of gene expression of Gene X, and estimated normal distribution and threshold value. No satisfactory separation is achieved when using this univariate classifier.

FIG. 4: Histogram of gene expression of Gene Y, and estimated normal distribution and threshold value. Again, no satisfactory separation is achieved when using a univariate classifier. In contrast to this, a bivariate classifier is able to separate groups A and B efficiently (cf. Example 4).

### DETAILED DESCRIPTION OF THE INVENTION

An “absolute expression level”, within the meaning of the invention, is understood as being the absolute expression level as obtained by using Affymetrix MAS5, which is well known to a person skilled in the art.

An “aggregate breast cancer response class”, within the meaning of the invention, shall be understood to be a breast cancer response class which comprises at least two sub-classes, each sub-class representing another aggregate or elementary breast cancer response class.

“Bivariate classification”, within the meaning of the invention, relates to the classification of breast cancer tumours into two or more (aggregate or elementary) breast cancer response classes, based on the expression levels of two marker genes. In the invention, this rather general mathematical notion is narrowed down to the special case of the determination of the bivariate normal distributions (expressed in terms of the mean vector and the covariance matrix) for the breast cancer response classes and the subsequent assignment of an unknown sample to the likeliest of said response classes by evaluating said normal distributions. Preferably, the bivariate classification comprises the determination of the bivariate normal distribution.

A “breast cancer response class” within the meaning of the invention, shall be understood to be a group of breast cancer tumours showing a similar gene expression pattern and/or similar clinical behaviour. Preferably, the members of a “breast cancer response class” show, or are likely to show, a similar response to chemotherapy. The gene expression pattern and/or the clinical behaviour is preferably not similar to the gene expression pattern and/or the clinical behaviour of other tumours which do not belong to said breast cancer response class, i.e. the tumours belonging to one breast cancer response class are preferably distinguishable from tumours not belonging to said class.

The terms “cancer” and “cancerous” refer to or describe the physiological condition in mammals that is typically characterized by unregulated cell growth.

“Chemotherapy”, within this context, is understood to be the treatment of cancer with cytotoxic drugs.

“Classification” within the meaning of the invention is understood to be the process of assigning a certain breast cancer response class to a given tumour. Classification can either be based on clinical information, or by applying a mathematical algorithm that utilizes clinical and/or gene expression data. Preferred classification methods of the invention are based on measurements of the expression of selected marker genes in a tumour sample.

A “correlation coefficient” between two variables, within the meaning of the invention, is understood to be the real number between −1 and 1 which measures the degree to which two variables are monotonely related. The correlation coefficient between two genes, within the context of the present application, shall be understood to be the correlation coefficient between the expression levels of said genes as determined in expression level measurements in multiple tissue samples. A high absolute correlation coefficient (i.e. negative signs disregarded) between two genes indicates that the two genes are co-regulated. In the following, correlation coefficient and correlation coefficient values shall be understood as being the absolute correlation coefficient values. A preferred correlation coefficient, within the context of the invention, is the “Pearson's Correlation Coefficient”.

“Determination of an expression level” of a gene in a tissue sample, within the meaning of the invention shall be understood to be any determination of the amount of mRNA coding for said gene, or a part of said gene, in said tissue sample; or any determination of the amount of the protein coded for by said gene in said tissue sample. Various methods to determine the expression level of a gene in a tissue are known in the art. These methods comprise, without limitation, PCR methods, real-time PCR methods, reverse transcriptase PCR methods, e.g. TaqMan RT-PCR, microarray experiments, immunohistochemistry (IHC), methods using the MassArray system of Sequenom, Inc. (San Diego, Calif.), SAGE Methods (Velculescu et al. 1995, Science 270, 484-487), the MPSS method of Brenner et al. (2000, Nature Biotechnology, 18, pp. 630-634) and other methods known to the person skilled in the art.

An “elementary breast cancer response class”, within the meaning of the invention, shall be understood to be a group of breast cancer tumours having similar expression levels of certain marker genes and/or similar clinical behaviour. Elementary breast cancer response classes preferably comprise no further distinct breast cancer response classes within.

A “marker gene”, within the meaning of the invention, is any gene, the expression level of which is useful for the classification of a tumour sample into one of several aggregate or elementary breast cancer response classes, according to the invention.

A “microarray” within the meaning of the invention, shall be understood as being any type of solid support material, comprising a multitude of local features, each feature comprising immobilized nucleic acid probes. These nucleic acid probes are able to bind to free nucleic acids in a sample, wherein such binding can be detected by suitable methods. Various suitable technical implementations of microarrays are known to the person skilled in the art and commercially available. One well known example of a microarray is the GeneChip™ of Affymetrix, Inc. (Santa Clara, Calif.).

“Neoadjuvant therapy”, within the meaning of the invention, is adjunctive or adjuvant therapy given prior to the primary (main) therapy. Neoadjuvant therapy includes, for example, chemotherapy, radiation therapy, and hormone therapy. Neoadjuvant chemotherapy, e.g., is administered prior to surgery to shrink the tumour, so that surgery can be more effective, or, in the case of previously inoperable tumours, can be made possible.

“Prediction of the response to chemotherapy”, within the meaning of the invention, shall be understood to be the act of determining a likely outcome of a chemotherapy in a patient inflicted with cancer. The prediction of a response is preferably made with reference to probability values for reaching a desired or non-desired outcome of the chemotherapy. The predictive methods of the present invention can be used clinically to make treatment decisions by choosing the most appropriate treatment modalities for any particular patient.

A “previously known characteristic property” of a breast cancer response class is a property common to tumours or individuals of this class. This property may relate, e.g., to their response to chemotherapeutic treatment. Preferably, a previously known characteristic property may be expressed in terms of a probability that a tumour or individual of a breast cancer response class shows a certain response to chemotherapy.

The term “prognosis” is used herein to refer to the prediction of the likelihood of cancer-attributable death or progression, including recurrence and metastatic spread, of a neoplastic disease, such as breast cancer.

The “response of a tumour to chemotherapy”, within the meaning of the invention, relates to any response of the tumour to chemotherapy, preferably to a change in tumour mass and/or volume after initiation of neoadjuvant chemotherapy. Tumour response may be assessed in a neoadjuvant situation where the size of a tumour after systemic intervention can be compared to the initial size and dimensions as measured by CT, PET, mammogram, ultrasound or palpation. Response may also be assessed by caliper measurement or pathological examination of the tumour after biopsy or surgical resection. Response may be recorded in a quantitative fashion like percentage change in tumour volume or in a qualitative fashion like “no change” (NC), “partial remission” (PR), “complete remission” (CR) or other qualitative criteria. Assessment of tumour response may be done early after the onset of neoadjuvant therapy e.g. after a few hours, days, weeks or preferably after a few months. A typical endpoint for response assessment is upon termination of neoadjuvant chemotherapy or upon surgical removal of residual tumour cells and/or the tumour bed. This is typically three month after initiation of neoadjuvant therapy.

A “tissue sample”, within the meaning of the invention, relates to tissue obtained from the human body by resection or biopsy which contains breast cancer cells. The tissue may originate from a carcinoma in situ, an invasive primary tumour, a recurrent tumour, lymph nodes infiltrated by tumour cells, or a metastatic lesion. The meaning of “tissue sample” is independent of the histological type of the primary tumour which may be an invasive ductal carcinoma, invasive lobular carcinoma, invasive tubular carcinoma, invasive medullar carcinoma, or invasive carcinoma of mixed type. After biopsy or resection, the breast tumour tissue may be preserved by storage in liquid nitrogen, dry ice or by fixation with appropriate reagents known in the field and subsequent embedding in paraffin wax. Preferably, tissue samples used in the present invention are already available, or are made available, prior to the start of the claimed methods. The detection of marker gene expression is not limited to the detection within a primary tumour, secondary tumour or metastatic lesion of breast cancer patients. It may also be detected in lymph nodes affected by breast cancer cells. In one embodiment of the invention, the sample to be analysed is tissue material from a neoplastic lesion taken by aspiration or punctuation, excision or by any other surgical method leading to biopsy or resected cellular material. The sample is preferably previously available. The step of taking the sample is preferably not part of the method. In one embodiment of the invention, the sample comprises cells obtained a breast cell “smear” collected, for example, by a nipple aspiration, ductal lavage, fine needle biopsy or from provoked or spontaneous nipple discharge. In another embodiment, the sample is a body fluid. Such fluids include, for example, blood fluids, lymph, ascitic fluids, gynecological fluids, or urine but not limited to these fluids.

The term “tumor,” as used herein, refers to all neoplastic cell growth and proliferation, whether malignant or benign, and all pre-cancerous and cancerous cells and tissues.

“Univariate classification”, within the meaning of the invention, is a classification of breast cancer tumours into two or more (aggregate or elementary) breast cancer response classes, based on the expression level of a single marker gene. Preferably, the classification comprises a comparison of the expression level of said marker gene with a predetermined threshold level.

Marker genes of the invention are defined either by their abbreviated gene name or by their ability to hybridise, i.e. to be detected, by probes defined in terms of their Affymetrix Probeset ID (see Table 4). Genes detected by a particular Affymetrix Probeset ID can be found at Affymetrix' homepage (http://www.affymetrix.com), or, more specific, at the HG U133A GeneChip Array Information Page on Affymetrix' homepage (http://www.affymetrix.com/support/technical/byproduct.affx?product=hgu133) and other sources known to the person skilled in the art.

The current invention relates to a method for the prediction of the response to chemotherapy of a breast cancer in a patient, from a sample of a tumour of said patient, comprising steps of
(a) determining the expression level of a group of marker genes consisting of
   (i) a first marker gene selected from the group consisting of MLPH, SPDEF, and AKR7A3; and
   (ii) a pair of second marker genes selected from the group of pairs consisting of (H2BFS and UBE2S), (BGN and ZBTB16), (ZBTB16 and EMP1), (LGALS8 and UBE2S) and (OLFML2B and ZBTB16); and
   (iii) a third marker gene selected from the group consisting of CYBA, ACP5, a gene specifically binding to Affymetrix probe set ID 210915_x_at, LCK, GSTM3;
(b) classifying said sample as belonging to one of several breast cancer response classes from the expression levels determined under (a);
(c) predicting the response of said breast cancer in said patient to chemotherapy from previously known characteristic properties of tumours of said one of several breast cancer response classes.

Methods of the invention use very small set of highly informative marker genes to classify a tumour sample as one out of several breast cancer response classes. It is envisaged, that the above combinations of marker genes represent the smallest possible groups of marker genes that allow classification of tumour samples into relevant breast cancer response classes.

The current invention further relates to a method of the above kind, wherein said several breast cancer response classes are four breast cancer response classes.

It is envisaged that four groups of breast cancer response classes are an optimal number of breast cancer response classes, because it allows for reliable classification and accurate prediction of the response of breast cancer tumours to EC-based chemotherapy.

The person skilled in the art will readily appreciate that it is possible to substitute the expression level of any of the marker genes of the invention by the expression level of a co-regulated gene, said substitute expression level holding the same information as the expression level of the original marker gene.

Hence, the current invention further relates to a method of the above kind, wherein at least one marker gene of said group of marker genes is substituted by a substitute marker gene, said substitute marker gene being co-regulated with said at least one marker gene.

Preferably, said substitute marker gene has a correlation coefficient to said at least one marker gene of equal to or higher than
(a) 0.816 in Table 1, if said marker gene is MLPH, SPDEF or AKR7A3;
(b) 0.827 in Table 2, if said marker gene is H2BFS, UBE2S, BGN, ZBTB16, EMP1, LGALS8 or OLFML2B; and
(c) 0.9013 in Table 3, if said marker gene is CYBA, ACP5, a gene specifically binding to Affymetrix probe set ID 210915_x_at, LCK or GSTM3.

It is envisaged, that these threshold values are appropriate for selecting substitute marker genes in methods of the invention. For calculation of these optimal threshold values, see Example 3.

Suitable substitute marker genes are identified by correlation coefficients listed in Tables 1-3, because this provides a measure which is well defined and utmostly independent of the test cohort used to determine the correlation coefficients. These correlation coefficients are highly significant by construction and so may be verified in separate experiments.

Alternatively, correlation coefficients determined from separate experiments can be used.

Alternative threshold values for the correlation coefficients in Tables 1-3 in methods of the invention are 0.7, 0.8, 0.816, 0.9, 0.95, 0.99, 0.999 or, most preferably 1.

Preferably, the classification step (b) in methods of the invention is based on a mathematical discriminant function or on a decision tree.

According to the invention, the classification scheme involves a decision tree with at least one bivariate classification step. The person skilled in the art will readily appreciate the advantages of the bivariate classification step, in certain cases, from Example 4.

Other preferred methods of the invention use a k-nearest-neighbour (kNN) algorithm in the classification step. Alternatively, classification can be achieved using i.a. the following mathematical methods: Decision Trees, Random Forests, (weighted) k-Nearest Neighbours, Shrunken Centroids, Support Vector Machines, Majority Votes, Neural Networks, Self-Organizing Maps (SOM), Cohonen Maps, Principal Curves and Principal Surfaces, Generative Topographic Mapping (GTM). These methods are widely used and readily available to the person skilled in the art.

In preferred methods of the invention, the chemotherapy is epirubicin/cyclophosphamide based chemotherapy.

In preferred methods of the invention, the chemotherapy is anthracyclines based chemotherapy.

In further preferred methods of the invention, the chemotherapy is a neoadjuvant chemotherapy.

Preferably, the predicted response to chemotherapy is a clinical response or a pathological response.

Patients in methods of the invention are preferably human patients.

According to the present invention, the sample of a tumour is preferably a fixed sample, a paraffin-embedded sample, a fresh sample, a fresh frozen sample or a frozen sample.

In a preferred embodiment of the invention, said sample of a tumour is from fine needle biopsy, core biopsy or fine needle aspiration.

In preferred methods of the invention, said determination of the expression level is by microarray experiment, by RT-PCR, by SAGE, by immunohistochemistry or by TaqMan.

The present invention further relates to a microarray comprising immobilized nucleic acid probes capable of specific hybridization with
a) a first marker gene selected from the group consisting of MLPH, SPDEF, and AKR7A3; and
b) two second marker genes in a pair selected from the group of pairs consisting of (H2BFS and UBE2S), (BGN and ZBTB16), (ZBTB16 and EMP1), (LGALS8 and UBE2S) and (OLFML2B and ZBTB16); and with
c) a third marker gene selected from the group consisting of CYBA, ACP5, a gene specifically binding to Affymetrix probe set ID 210915_x_at, LCK, GSTM3.

Specific hybridization on a microarray, within the meaning of the invention, means hybridization between a nucleic acid in a sample and an immobilized nucleic acid probe on the array, which occurs under conditions typically applied in microarray experiments, preferably under conditions which are recommended by the producer of the microarray or microarray system.

Preferred microarrays of the invention are RNA arrays or DNA arrays.

The invention further relates to a system for predicting the response of a breast cancer in a patient to chemotherapy, comprising
(a) means for determining the expression level of a group of marker genes consisting of
   (i) a first marker gene selected from the group consisting of MLPH, SPDEF, and AKR7A3; and
   (ii) a pair of second marker genes selected from the group of pairs consisting of (H2BFS and UBE2S), (BGN and ZBTB16), (ZBTB16 and EMP1), (LGALS8 and UBE2S) and (OLFML2B and ZBTB16); and
   (iii) a third marker gene selected from the group consisting of CYBA, ACP5, a gene specifically binding to Affymetrix probe set ID 210915_x_at, LCK, GSTM3.
(b) computing means adapted for classifying said sample to one of several breast cancer response classes from expression levels of said group of marker genes,
(c) computing means adapted for predicting the response of said breast cancer in said patient to chemotherapy from characteristic properties of tumours of said one of several breast cancer response class.

Preferred systems of the invention classify a sample into one of four (4) breast cancer response classes.

Preferred systems of the invention comprise means for determining the expression level of a group of marker genes being a microarray, a system for 2D gel electrophoresis, a SAGE system or a system for immunohistochemical determination of expression levels.

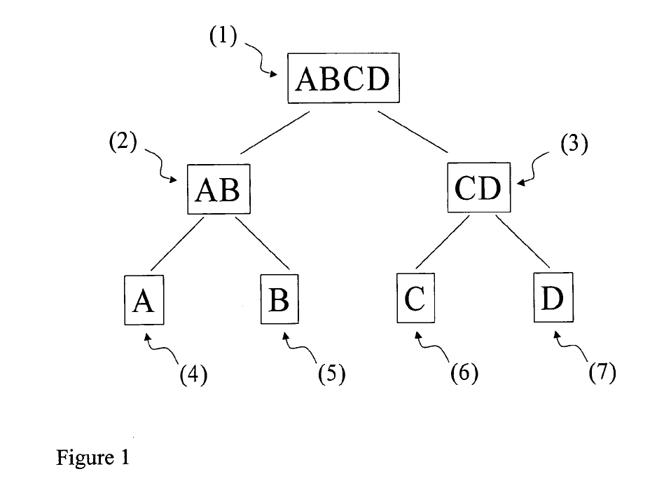

Preferred methods of the invention are methods comprising the steps of
(a) determining the expression level of at least one first marker gene in said sample of said tumour;
(b) classifying said sample as belonging to a first (FIG. 1, reference numeral 2) or a second (reference numeral 3) aggregate breast cancer response class from the expression level of said at least one first marker gene,
(c) determining the expression level of at least one second marker gene;
(d) classifying said sample as belonging to a first (4, 6) or a second (5, 7) elementary breast cancer response class of said first (2) or second (3) aggregate breast cancer response class from said expression level of said at least one second marker gene; and
(e) predicting the response of said breast cancer in said patient to chemotherapy from previously known characteristic properties of tumours of said first (4, 6) or second (5, 7) elementary breast cancer response class of said first (2) or second (3) aggregate breast cancer response class,
   wherein the choice of said at least one second marker gene is specific for (or alternatively, is dependent on) the aggregate breast cancer response class determined in step b).

The invention further relates to a method for the classification of a breast cancer tumour into clinically relevant breast cancer response classes, said method comprising steps of (a) determining the expression level of at least one first marker gene in said sample of said tumour; (b) classifying said sample as belonging to a first (2) or a second (3) aggregate breast cancer response class from the expression level of said at least one first marker gene, (c) determining the expression level of at least one second marker gene; and (d) classifying said sample as belonging to a first (4, 6) or a second (5, 7) elementary breast cancer response class of said first (2) or second (3) aggregate breast cancer response class from said expression level of said at least one second marker gene, wherein the choice of said at least one second marker gene is specific for the aggregate breast cancer response class determined in step b).

In a preferred embodiment of the invention, the expression levels are determined with RT-PCR, on a microarray, or by quantification of the protein encoded by the measured gene, e.g. by 2 dimensional gel electrophoresis a system for immunohistochemical determination of the expression level.

According to a preferred embodiment of the invention, the step of determining the expression level of a marker gene is performed ex vivo.

Preferably, all method steps above are performed ex vivo. Furthermore, preferred methods comprise only method steps which are not performed on the human or animal body. Particularly preferred methods do not require the presence of the patient in any step of the method.

Determination of the expression levels of said at least one first and second marker gene is preferably done in parallel e.g. on a microarray.

In a preferred method of the invention, said first classification step (b) is a univariate classification.

In preferred methods of the invention, the at least one first marker gene is MLPH, SPDEF, AKR7A3 or, optionally, a gene having a correlation coefficient to MLPH, SPDEF or AKR7A3 which is equal to or exceeding 0.816 in Table 1 (cf. Table 4 for identification of the gene). Any of said at least one first marker genes can be used individually in the methods of the invention. It is, however, also possible to use more than one of said marker genes and to perform a classification on the basis of multiple expression level measurements. Measuring a single first marker gene, however, is preferred.

The threshold value for the correlation coefficient in Table 1 in methods of the invention is preferably 0.7, 0.8, 0.816, 0.9, 0.95, 0.99, 0.999 or, most preferably 1. In preferred embodiments of the invention the threshold value is one employed in Example 2. Alternatively, a suitable correlation coefficient can be determined in a separate expression profiling experiment, involving multiple tissue samples.

The invention also relates to a method as defined above in which the tumour is classified as belonging to said first aggregate breast cancer response class (2) if the expression of said at least one first marker gene exceeds a predetermined threshold value, and wherein the tumour is classified as belonging to said second aggregate breast cancer response class (3) if the expression of said at least one first marker gene is equal to or below said predetermined threshold value. In preferred methods of the invention, the threshold value for the expression level of said at least one first marker gene is preferably identified from previous experiments. This threshold value is such that its application in a method of the inventions allows a meaningful separation of the tumours into two aggregate breast cancer response classes (2, 3).

In preferred methods of the invention, the second classification step (d) is a univariate or a bivariate classification. Univariate classification is preferred in cases in which a single marker gene provides good or sufficient separation of the tumours into the first and second aggregate breast cancer response class. Bivariate classification is used in cases where a single marker gene does not provide good or sufficient separation of the tumours into the first and second aggregate breast cancer response class.

In preferred embodiments of the invention, a bivariate classifier is used to separate the first aggregate breast cancer response class (2) into the first (4) and second (5) elementary breast cancer response class of said first aggregate breast cancer response class (2). Preferably, a univariate classifier is used to separate the first (6) and second (7) elementary breast cancer response class from the second (3) aggregate breast cancer response class.

In another embodiment of the method, in-class probabilities are estimated by the predictor, giving not only the most probable class but also information about the likeliness of alternative class predictions. One embodiment of the method uses a hierarchical binary classification technique (n=2) in each node. This preferably involves the computation of the in-class-probability for each sample to each class. In another embodiment, the approach is able to cope with an arbitrary number of classes (n>2) at the same time. The set of partial classifiers builds the global classifier. The number of marker genes used in each partial classifier can be as low as 1 or 2, but also larger numbers of genes may be used.

In preferred methods of the invention, if said sample was classified as belonging to said first aggregate breast cancer response class (2), i.e. class “B”, said at least one second marker gene is a pair of marker genes selected from the group consisting of (H2BFS and UBE2S), (BGN and ZBTB16), (ZBTB16 and EMP1), (LGALS8 and UBE2S) and (OLFML2B and ZBTB16) and pairs of marker genes having a correlation coefficients to the first and second member of said pairs, respectively, which are equal to or exceeding 0.827 in Table 1.

In preferred methods of the invention, if said sample was classified as belonging to said second aggregate breast cancer response class (3), said at least one second marker gene is CYBA, ACP5, a gene specifically binding to Affymetrix probe set ID 210915_x_at, LCK, GSTM3 or, optionally, a gene having a correlation coefficient to CYBA, ACP5, a gene specifically binding to Affymetrix probe set ID 210915_x_at, LCK, GSTM3, which is equal to or exceeding 0.9013 in Table 1.

In preferred methods of the invention, the chemotherapy is an EC-based chemotherapy.

In preferred methods of the invention, the chemotherapy is anthracyclines based chemotherapy.

In preferred methods of the invention, the chemotherapy is a neoadjuvant chemotherapy.

In preferred methods of the invention, if said tumour is classified to belong to the first elementary tumour class (4) of the first aggregate tumour class (2), the tumour is predicted to have a low likelihood of “pathological complete response” (i.e. 100% reduction in tumour mass), a low likelihood of “good partial response” (i.e. >75% reduction in tumour mass), an intermediate likelihood of partial response (a reduction in tumour mass of >25% but <75%), an intermediate likelihood of bad partial response (a reduction in tumour mass of >0% but <25% and an intermediate likelihood of “no response” (i.e. no reduction in tumour mass), upon neoadjuvant EC-based chemotherapy.

In preferred methods of the invention, if said tumour is classified to belong to the second elementary tumour class (5) of the first aggregate tumour class (2), the tumour is predicted to have a low likelihood of “pathological complete response”, a intermediate likelihood of “good partial response”, a low likelihood of “partial response”, a low likelihood of “bad partial response” and a low likelihood of “no response”, upon neoadjuvant EC treatment.

In preferred methods of the invention, if said tumour is classified to belong to the first elementary tumour class (6) of the second aggregate tumour class (3), the tumour is predicted to have a high likelihood of “pathological complete response”, a low likelihood of “good partial response”, a low likelihood of “partial response”, a low likelihood of “bad partial response” and a low likelihood of “no response”, upon neoadjuvant EC treatment.

In preferred methods of the invention, if said tumour is classified to belong to the second elementary tumour class (7) of the second aggregate tumour class (3), the tumour is predicted to have a low likelihood of “pathological complete response”, a low likelihood of “good partial response”, an intermediate likelihood of “partial response”, a low likelihood of “bad partial response” and a low likelihood of “no response”, upon neoadjuvant EC treatment.

A “low likelihood”, within the meaning of the invention, is preferably a likelihood p with 0≦p<25%. A “intermediate likelihood”, within the meaning of the invention, is a likelihood p with 25%≦p<75%. A “high likelihood”, within the meaning of the invention, is a likelihood p with 75%≦p<100%.

Another aspect of the invention relates to methods for treating breast cancer in a patient, said method comprising one of the above methods of predicting the response of a breast cancer to chemotherapy, and applying said chemotherapy, if said breast cancer is predicted to show a sufficiently good response to said chemotherapy. A “sufficiently good response”, in this case, shall be a likelihood for pathological complete response of >20%, >50%, >80%, >90%, >95%, preferably >99%. According to another aspect of the invention, a “sufficiently good response” shall be understood as being a likelihood for good partial response of >20%, >50%, >80%, >90%, >95%, preferably >99%. “Sufficiently good response” may also be a likelihood for partial response of >20%, >50%, >80%, >90%, >95%, preferably >99%.

The invention furthermore relates to kits for use in methods of the invention. Such kits comprise means for the determination of the expression level of said at least one first marker gene and means for the determination of the expression level of said at least one second marker gene. These means are preferably microarrays or a selection of reagents required for RT-PCR. Preferably, kits of the invention furthermore comprise computing means for the automatic processing of the determined expression levels, such as a micro-controller or a computer. Computing means according to the invention are able to automatically select appropriate second marker genes for the second classification step in methods of the invention. Kits of the invention advantageously comprise display means for displaying the identified tumour class and storage means for storing expression data and other patient related data.

The invention is further illustrated by way of the following examples. It shall be understood that the invention is not restricted to the specific embodiments described in the examples hereinafter.

### EXAMPLES

### Example 1

### Patient Selection, RNA Isolation from Tumour Tissue Biopsies and Gene Expression Measurement Utilizing HG-U133A Arrays of Affymetrix

Samples of primary breast carcinomas were available from 80 patients subjected to neoadjuvant treatment with epirubicin/cyclophosphamide (EC). EC consisted of epirubicin 90 mg m² per day 1 in a short i.v. infusion, and cyclophosphamide 600 mg m² per day 1 in a short i.v. infusion. Four cycles of EC were administrated 14 days apart. All tumour samples were collected as needle biopsies of primary tumours prior to any treatment. The biopsies were obtained under local anaesthesia using Bard® MAGNUM™ Biopsy Instrument (C.R. Bard, Inc., Covington, US) with Bard® Magnum biopsy needles (BIP GmbH, Tuerkenfeld, Germany) following ultrasound guidance.

Total RNA was isolated from snap frozen breast tumour tissue biopsies. The tissue was crushed in liquid nitrogen, RLT-Buffer (QIAGEN, Hilden, Germany) was added and the homogenate spun through a QIAshredder column (QIAGEN, Hilden, Germany). From the eluate total RNA was isolated by the RNeasy Kit (QIAGEN, Hilden, Germany) according to the manufacturers instruction. RNA yield was determined by UV absorbance and RNA quality was assessed by analysis of ribosomal RNA band integrity on the Agilent Bioanalyzer (Palo Alto, Calif., USA).

Starting from 5 μg total RNA labelled cRNA was prepared for all 80 tumour samples using the one-cycle target labelling kit together with the appropriate control reagents (Affymetrix, Santa Clara, Calif., USA) according to the manufacturer's instruction. In brief, synthesis of first strand cDNA was done by a T7-linked oligo-dT primer, followed by second strand synthesis. Double-stranded cDNA product was purified and then used as template for an in vitro transcription reaction (IVT) in the presence of biotinylated UTP. Labelled cRNA was hybridised to HG-U133A arrays (Affymetrix, Santa Clara, Calif., USA) at 45° C. for 16 h in a hybridisation oven at a constant rotation (60 r.p.m.) and then washed and stained with a streptavidin-phycoerythrin conjugate using the GeneChip fluidic station. We scanned the arrays at 560 nm using the GeneArray Scanner G2500A from Hewlett Packard. The readings from the quantitative scanning were analysed using the Microarray Analysis Suit 5.0 (MAS 5.0) from Affymetrix. In the analysis settings the global scaling procedure was chosen which multiplied the output signal intensities of each array to a mean target intensity of 500. Routinely we obtained over 50 percent present calls per chip as calculated by MAS 5.0.

### Example 2

### Classification of Breast Tumour Tissues into EC Response Classes

For the separation of the aggregate breast cancer response classes AB and CD from ABCD (cf. FIG. 1) one of the following partial classifiers is used:
1. A univariate classification based on a single gene expression is provided by measuring the expression level of MLPH (Affymetrix Probe Set ID 218211_s_at) and comparing it with a threshold value of 1733. Samples with a higher expression of MLPH compared to the threshold value are aggregate breast cancer response class AB, whereas such with a lower expression are aggregate breast cancer response class CD.
2. Alternatively, the expression level of SPDEF (Affymetrix Probe Set ID 213441_x_at) is compared with a threshold of 1091, SPDEF (214404_x_at) with a threshold of 626, SPDEF (220192_x_at) with a threshold of 867, or AKR7A3 (216381_x_at) with a threshold of 402. In each of these cases, samples with an expression higher than the corresponding threshold are class AB, samples with an expression lower than the threshold are CD.

TABLE 1
Correlation coefficients of correlated genes in classification AB <-> CD

| Correlate | | | | | | | |
|---|---|---|---|---|---|---|---|
| gene name | | Genes covered in the examples | | | | | |
| | Gene | AKR7A3 | MLPH | SPDEF | SPDEF | SPDEF | EMP1 |
| Nr. | Symbol | (216381_x_at) | (218211_s_at) | (213441_x_at) | (214404_x_at) | (220192_x_at) | (201324_at) |
| 1 | GATA3 | 0.73 | 0.86 | 0.77 | 0.75 | 0.76 | 0.07 |
| 2 | MLPH | 0.71 | 1.00 | 0.79 | 0.80 | 0.81 | 0.02 |
| 3 | AGR2 | 0.71 | 0.84 | 0.73 | 0.72 | 0.69 | −0.07 |
| 4 | VAV3 | 0.64 | 0.84 | 0.72 | 0.72 | 0.72 | 0.04 |
| 5 | SPDEF | 0.73 | 0.79 | 1.00 | 0.92 | 0.93 | −0.07 |
| 6 | SPDEF | 0.73 | 0.81 | 0.93 | 0.94 | 1.00 | −0.08 |
| 7 | PH-4 | 0.73 | 0.84 | 0.72 | 0.73 | 0.77 | −0.06 |
| 8 | MYB | 0.59 | 0.84 | 0.68 | 0.67 | 0.67 | 0.02 |
| 9 | GATA3 | 0.67 | 0.87 | 0.71 | 0.74 | 0.73 | −0.02 |
| 10 | SPDEF | 0.66 | 0.80 | 0.92 | 1.00 | 0.94 | −0.15 |
| 11 | FOXA1 | 0.71 | 0.90 | 0.78 | 0.85 | 0.84 | −0.03 |
| 12 | GATA3 | 0.63 | 0.84 | 0.70 | 0.70 | 0.70 | 0.11 |
| 13 | C6orf29 | 0.70 | 0.83 | 0.78 | 0.76 | 0.82 | −0.04 |
| 14 | AKR7A3 | 1.00 | 0.71 | 0.73 | 0.66 | 0.73 | −0.06 |
| 15 | AKR7A3 | 0.95 | 0.71 | 0.72 | 0.66 | 0.74 | −0.05 |
| 16 | LOC400451 | 0.68 | 0.70 | 0.79 | 0.76 | 0.82 | 0.07 |
| 17 | FOXC1 | −0.70 | −0.74 | −0.76 | −0.76 | −0.83 | 0.14 |

| Correlate | | | | | | | |
|---|---|---|---|---|---|---|---|
| gene name | | Genes covered in the examples | | | | | |
| | Gene | GSTM3 | UBE2S | CYBA | ACP5 | LCK | |
| Nr. | Symbol | (202554_s_at) | (202779_s_at) | (203028_s_at) | (204638_at) | (204891_s_at) | ZBTB16 (205883_at) |
| 1 | GATA3 | 0.44 | −0.49 | −0.23 | −0.01 | −0.33 | 0.53 |
| 2 | MLPH | 0.52 | −0.48 | −0.29 | −0.10 | −0.24 | 0.44 |
| 3 | AGR2 | 0.38 | −0.39 | −0.16 | −0.02 | −0.26 | 0.40 |
| 4 | VAV3 | 0.54 | −0.47 | −0.20 | −0.03 | −0.21 | 0.44 |
| 5 | SPDEF | 0.57 | −0.32 | −0.12 | 0.06 | −0.20 | 0.37 |
| 6 | SPDEF | 0.54 | −0.31 | −0.12 | 0.07 | −0.19 | 0.35 |
| 7 | PH-4 | 0.56 | −0.39 | −0.27 | 0.06 | −0.24 | 0.41 |
| 8 | MYB | 0.42 | −0.41 | −0.17 | 0.04 | −0.16 | 0.39 |
| 9 | GATA3 | 0.42 | −0.41 | −0.29 | 0.02 | −0.30 | 0.48 |
| 10 | SPDEF | 0.52 | −0.25 | −0.06 | 0.09 | −0.11 | 0.36 |
| 11 | FOXA1 | 0.48 | −0.38 | −0.21 | 0.00 | −0.20 | 0.36 |
| 12 | GATA3 | 0.41 | −0.42 | −0.30 | 0.00 | −0.35 | 0.43 |
| 13 | C6orf29 | 0.44 | −0.40 | −0.19 | −0.04 | −0.21 | 0.41 |
| 14 | AKR7A3 | 0.39 | −0.34 | −0.05 | 0.14 | −0.19 | 0.25 |
| 15 | AKR7A3 | 0.40 | −0.32 | −0.12 | 0.13 | −0.25 | 0.27 |
| 16 | LOC400451 | 0.44 | −0.34 | −0.22 | 0.04 | −0.25 | 0.42 |
| 17 | FOXC1 | −0.50 | 0.24 | 0.04 | −0.11 | 0.04 | −0.32 |

| Correlate | | | | | | |
|---|---|---|---|---|---|---|
| gene name | | Genes covered in the examples | | | | |
| | Gene | H2BFS | LGALS8 | TRBV19 /// TRBC1 | | BGN /// SDCCAG33 |
| Nr. | Symbol | (208579_x_at) | (208933_s_at) | (210915_x_at) | OLFML2B (213125_at) | (213905_x_at) |
| 1 | GATA3 | −0.16 | 0.57 | −0.28 | 0.34 | 0.22 |
| 2 | MLPH | −0.08 | 0.69 | −0.23 | 0.28 | 0.13 |
| 3 | AGR2 | −0.01 | 0.59 | −0.14 | 0.16 | 0.10 |
| 4 | VAV3 | −0.13 | 0.55 | −0.23 | 0.26 | 0.15 |
| 5 | SPDEF | −0.11 | 0.41 | −0.16 | 0.26 | 0.18 |
| 6 | SPDEF | −0.07 | 0.46 | −0.11 | 0.29 | 0.19 |
| 7 | PH-4 | −0.02 | 0.52 | −0.16 | 0.30 | 0.19 |
| 8 | MYB | −0.16 | 0.70 | −0.16 | 0.27 | 0.15 |
| 9 | GATA3 | −0.13 | 0.59 | −0.25 | 0.26 | 0.16 |
| 10 | SPDEF | −0.08 | 0.41 | −0.04 | 0.22 | 0.11 |
| 11 | FOXA1 | −0.04 | 0.62 | −0.13 | 0.29 | 0.10 |
| 12 | GATA3 | −0.17 | 0.64 | −0.32 | 0.28 | 0.17 |
| 13 | C6orf29 | −0.01 | 0.65 | −0.09 | 0.30 | 0.11 |
| 14 | AKR7A3 | 0.01 | 0.33 | −0.07 | 0.34 | 0.22 |
| 15 | AKR7A3 | 0.05 | 0.32 | −0.13 | 0.32 | 0.26 |
| 16 | LOC400451 | −0.18 | 0.45 | −0.18 | 0.23 | 0.19 |
| 17 | FOXC1 | 0.05 | −0.49 | −0.06 | −0.19 | −0.13 |

For the subsequent separation of the elementary breast cancer response classes A and B, the following partial classifier was used:
1. The gene expression level of one or more genes used in the partial classifiers are measured for each tumour sample.
2. With g₁ being the binary (base 2) logarithm of the absolute expression level of H2BFS (208579_x_at) and g₂ being the binary logarithm of the absolute expression level of UBE2S (202779_s_at), evaluate:

$${p}_{1} := \frac{1}{\sqrt{{( 2 \cdot π )}^{2} \cdot \vert det {Σ}_{1} \vert}} \cdot exp ( - \frac{1}{2} \cdot {( g - {μ}_{1} )}^{t} {Σ}_{1}^{- 1} ( g - {μ}_{1} ) )$$

$${p}_{2} := \frac{1}{\sqrt{{( 2 \cdot π )}^{2} \cdot \vert det {Σ}_{2} \vert}} \cdot exp ( - \frac{1}{2} \cdot {( g - {μ}_{2} )}^{t} {Σ}_{2}^{- 1} ( g - {μ}_{2} ) )$$

$$with$$

$$g := ( \begin{matrix}{g}_{1} \\ {g}_{2}\end{matrix} ) , {μ}_{1} := ( \begin{matrix}9.76 \\ 9.52\end{matrix} ) , {μ}_{2} := ( \begin{matrix}10.58 \\ 11.20\end{matrix} ) , \\ {Σ}_{1} := ( \begin{matrix}3.27 & - 0.551 \\ - 0.551 & 0.362\end{matrix} ) , {Σ}_{2} := ( \begin{matrix}0.963 & 0.404 \\ 0.404 & 0.474\end{matrix} )$$

If p₁>p₂, we assign the tumour to the first elementary class (A) of the first aggregate class (AB). Otherwise, the tumour is in class B.
3. Another possible classifier is the following bivariate classifier: With g₁ being the binary (base 2) logarithm of the absolute expression level of BGN (213905_x_at) and g₂ being the binary logarithm of the absolute expression level of ZBTB16 (205883_at), evaluate

$${p}_{1} := \frac{1}{\sqrt{{( 2 \cdot π )}^{2} \cdot \vert det {Σ}_{1} \vert}} \cdot exp ( - \frac{1}{2} \cdot {( g - {μ}_{1} )}^{t} {Σ}_{1}^{- 1} ( g - {μ}_{1} ) )$$

$${p}_{2} := \frac{1}{\sqrt{{( 2 \cdot π )}^{2} \cdot \vert det {Σ}_{2} \vert}} \cdot exp ( - \frac{1}{2} \cdot {( g - {μ}_{2} )}^{t} {Σ}_{2}^{- 1} ( g - {μ}_{2} ) )$$

$$with$$

$$g := ( \begin{matrix}{g}_{1} \\ {g}_{2}\end{matrix} ) , {μ}_{1} := ( \begin{matrix}11.71 \\ 8.33\end{matrix} ) , {μ}_{2} := ( \begin{matrix}10.37 \\ 6.68\end{matrix} ) , \\ {Σ}_{1} := ( \begin{matrix}0.622 & - 0.138 \\ - 0.138 & 0.669\end{matrix} ) , {Σ}_{2} := ( \begin{matrix}0.862 & - 0.291 \\ - 0.291 & 0.324\end{matrix} )$$

If p₁>p₂, we assign the unknown sample to the first class, A, and if not, to the second class, B.
4. Another example for such a classifier is the following bivariate classifier: With g₁ being the binary (base 2) logarithm of the absolute expression level of ZBTB16 (205883_at_x_at) and being the binary logarithm of the absolute expression level of EMP1 (201324_at), evaluate

$${p}_{1} := \frac{1}{\sqrt{{( 2 \cdot π )}^{2} \cdot \vert det {Σ}_{1} \vert}} \cdot exp ( - \frac{1}{2} \cdot {( g - {μ}_{1} )}^{t} {Σ}_{1}^{- 1} ( g - {μ}_{1} ) )$$

$${p}_{2} := \frac{1}{\sqrt{{( 2 \cdot π )}^{2} \cdot \vert det {Σ}_{2} \vert}} \cdot exp ( - \frac{1}{2} \cdot {( g - {μ}_{2} )}^{t} {Σ}_{2}^{- 1} ( g - {μ}_{2} ) )$$

$$with$$

$$g := ( \begin{matrix}{g}_{1} \\ {g}_{2}\end{matrix} ) , {μ}_{1} := ( \begin{matrix}8.33 \\ 10.24\end{matrix} ) , {μ}_{2} := ( \begin{matrix}6.68 \\ 9.34\end{matrix} ) , \\ {Σ}_{1} := ( \begin{matrix}0.668 & - 0.0933 \\ - 0.0 .0933 & 0.399\end{matrix} ) , {Σ}_{2} := ( \begin{matrix}0.324 & - 0.495 \\ - 0.495 & 0.960\end{matrix} )$$

If p₁>p₂, we assign the unknown sample to the first class, A, and if not, to the second class, B.
5. Another example for such a classifier is the following bivariate classifier: With g₁ being the binary (base 2) logarithm of the absolute expression level of LGALS8 (208933_s_at) and g₂ being the binary logarithm of the absolute expression level of UBE2S (202779_s_at), evaluate

$${p}_{1} := \frac{1}{\sqrt{{( 2 \cdot π )}^{2} \cdot \vert det {Σ}_{1} \vert}} \cdot exp ( - \frac{1}{2} \cdot {( g - {μ}_{1} )}^{t} {Σ}_{1}^{- 1} ( g - {μ}_{1} ) )$$

$${p}_{2} := \frac{1}{\sqrt{{( 2 \cdot π )}^{2} \cdot \vert det {Σ}_{2} \vert}} \cdot exp ( - \frac{1}{2} \cdot {( g - {μ}_{2} )}^{t} {Σ}_{2}^{- 1} ( g - {μ}_{2} ) )$$

$$with$$

$$g := ( \begin{matrix}{g}_{1} \\ {g}_{2}\end{matrix} ) , {μ}_{1} := ( \begin{matrix}9.94 \\ 9.52\end{matrix} ) , {μ}_{2} := ( \begin{matrix}9.95 \\ 11.2\end{matrix} ) , \\ {Σ}_{1} := ( \begin{matrix}0.493 & - 0.149 \\ - 0.149 & 0.362\end{matrix} ) , {Σ}_{2} := ( \begin{matrix}0.984 & - 0.438 \\ - 0.438 & 0.474\end{matrix} )$$

If p₁>p₂, we assign the unknown sample to the first class, A, and if not, to the second class, B.
6. Another example for such a classifier is the following bivariate classifier: With g₁ being the binary (base 2) logarithm of the absolute expression level of OLFML2B (213125_at) and g₂ being the binary logarithm of the absolute expression level of ZBTB16 (205883_at), evaluate

$${p}_{1} := \frac{1}{\sqrt{{( 2 \cdot π )}^{2} \cdot \vert det {Σ}_{1} \vert}} \cdot exp ( - \frac{1}{2} \cdot {( g - {μ}_{1} )}^{t} {Σ}_{1}^{- 1} ( g - {μ}_{1} ) )$$

$${p}_{2} := \frac{1}{\sqrt{{( 2 \cdot π )}^{2} \cdot \vert det {Σ}_{2} \vert}} \cdot exp ( - \frac{1}{2} \cdot {( g - {μ}_{2} )}^{t} {Σ}_{2}^{- 1} ( g - {μ}_{2} ) )$$

$$with$$

$$g := ( \begin{matrix}{g}_{1} \\ {g}_{2}\end{matrix} ) , {μ}_{1} := ( \begin{matrix}10.61 \\ 8.33\end{matrix} ) , {μ}_{2} := ( \begin{matrix}9.53 \\ 6.68\end{matrix} ) , \\ {Σ}_{1} := ( \begin{matrix}0.554 & - 0.237 \\ - 0.237 & 0.669\end{matrix} ) , {Σ}_{2} := ( \begin{matrix}0.782 & - 0.274 \\ - 0.274 & 0.324\end{matrix} )$$

If p₁>p₂, we assign the unknown sample to the first class, A, and if not, to the second class, B.

TABLE 2
Correlation coefficients of genes co-regulated with preferred marker genes for separation of
classes A and B from aggregate class AB

| Correlate gene | | Genes covered in the examples | | | | | |
|---|---|---|---|---|---|---|---|
| | Gene | AKR7A3 | MLPH | SPDEF | SPDEF | SPDEF | EMP1 |
| Nr. | Symbol | (216381_x_at) | (218211_s_at) | (213441_x_at) | (214404_x_at) | (220192_x_at) | (201324_at) |
| 1 | COL1A2 | 0.16 | −0.09 | −0.05 | −0.06 | −0.17 | 0.55 |
| 2 | COL1A1 | 0.14 | −0.06 | −0.06 | −0.11 | −0.18 | 0.54 |
| 3 | COL3A1 | 0.12 | −0.02 | −0.29 | −0.28 | −0.33 | 0.51 |
| 4 | COL1A2 | 0.08 | −0.03 | −0.12 | −0.15 | −0.23 | 0.64 |
| 5 | SPARC | 0.16 | −0.09 | −0.10 | −0.13 | −0.19 | 0.63 |
| 6 | COL6A1 | 0.08 | −0.04 | −0.10 | −0.15 | −0.20 | 0.54 |
| 7 | COL6A2 | 0.10 | −0.09 | −0.09 | −0.15 | −0.15 | 0.54 |
| 8 | CSPG2 | 0.03 | −0.03 | −0.06 | −0.12 | −0.15 | 0.68 |
| 9 | DKFZp564I1922 | 0.06 | −0.06 | −0.10 | −0.12 | −0.23 | 0.68 |
| 10 | COL5A2 | 0.07 | −0.08 | −0.18 | −0.23 | −0.25 | 0.71 |
| 11 | FSTL1 | 0.05 | −0.02 | −0.21 | −0.26 | −0.28 | 0.64 |
| 12 | KIAA0992 | −0.05 | 0.09 | −0.16 | −0.26 | −0.27 | 0.66 |
| 13 | CSPG2 | 0.04 | −0.01 | −0.14 | −0.21 | −0.20 | 0.64 |
| 14 | PRSS11 | 0.08 | −0.01 | −0.23 | −0.27 | −0.28 | 0.61 |
| 15 | THBS2 | 0.04 | −0.09 | −0.10 | −0.18 | −0.19 | 0.63 |
| 16 | FBN1 | 0.10 | −0.06 | −0.20 | −0.25 | −0.27 | 0.64 |
| 17 | COL5A2 | 0.15 | −0.09 | −0.31 | −0.36 | −0.33 | 0.56 |
| 18 | SPARC | 0.03 | −0.06 | −0.26 | −0.26 | −0.32 | 0.65 |
| 19 | COL5A1 | 0.15 | −0.08 | −0.08 | −0.16 | −0.17 | 0.59 |
| 20 | AEBP1 | 0.05 | −0.24 | −0.09 | −0.16 | −0.12 | 0.59 |
| 21 | BGN /// | 0.10 | −0.06 | 0.05 | −0.04 | −0.06 | 0.61 |
| | SDCCAG33 | | | | | | |
| 22 | CDH11 | 0.03 | −0.03 | −0.30 | −0.36 | −0.31 | 0.71 |
| 23 | BGN | 0.12 | 0.00 | 0.03 | −0.06 | −0.07 | 0.58 |
| 24 | MGC3047 | 0.11 | −0.01 | −0.06 | −0.12 | −0.17 | 0.56 |
| 25 | ASPN | −0.01 | 0.02 | −0.15 | −0.18 | −0.26 | 0.60 |
| 26 | LRRC15 | 0.08 | −0.05 | −0.16 | −0.21 | −0.29 | 0.55 |
| 27 | COL5A1 | 0.12 | −0.09 | −0.23 | −0.30 | −0.30 | 0.62 |
| 28 | DCN | −0.06 | −0.09 | −0.21 | −0.20 | −0.25 | 0.75 |
| 29 | COL5A1 | 0.15 | −0.14 | −0.20 | −0.27 | −0.26 | 0.57 |
| 30 | DPYSL3 | −0.04 | −0.08 | −0.10 | −0.15 | −0.19 | 0.64 |
| 31 | PCOLCE | 0.18 | −0.21 | −0.14 | −0.20 | −0.16 | 0.49 |
| 32 | MRC2 | 0.05 | 0.03 | −0.05 | −0.15 | −0.20 | 0.54 |
| 33 | H2BFS | 0.19 | 008 | −0.11 | −0.14 | −0.04 | −0.35 |
| 34 | OLFML2B | 0.07 | 0.01 | −0.14 | −0.18 | −0.19 | 0.58 |
| 35 | UBE2S | 0.00 | −0.37 | −0.02 | 0.05 | 0.08 | −0.55 |
| 36 | EMP1 | −0.17 | 0.05 | −0.23 | −0.26 | −0.26 | 1.00 |
| 37 | FAP | 0.05 | −0.02 | −0.30 | −0.37 | −0.34 | 0.66 |
| 38 | SPON1 | −0.02 | −0.05 | −0.22 | −0.26 | −0.32 | 0.63 |
| 39 | LGALS8 | −0.25 | 0.59 | −0.12 | −0.16 | −0.06 | 0.31 |
| 40 | LOC83468 | 0.02 | −0.02 | −0.34 | −0.34 | −0.36 | 0.62 |
| 41 | COL8A2 | 0.08 | −0.11 | −0.32 | −0.36 | −0.35 | 0.58 |
| 42 | SDC2 | −0.01 | 0.11 | −0.14 | −0.16 | −0.19 | 0.43 |
| 43 | PDGFRL | −0.03 | −0.08 | −0.20 | −0.18 | −0.22 | 0.58 |
| 44 | C1QTNF3 | −0.07 | 0.09 | −0.21 | −0.27 | −0.29 | 0.54 |
| 45 | SPON1 | 0.05 | −0.02 | −0.32 | −0.32 | −0.33 | 0.48 |
| 46 | OMD | −0.04 | −0.04 | −0.25 | −0.22 | −0.28 | 0.55 |
| 47 | SPON1 | 0.00 | −0.03 | −0.21 | −0.25 | −0.28 | 0.70 |
| 48 | ZBTB16 | −0.13 | 0.26 | 0.21 | 0.26 | 0.07 | 0.13 |

| Correlate gene | | Genes covered in the examples | | | | | |
|---|---|---|---|---|---|---|---|
| | Gene | GSTM3 | UBE2S | CYBA | ACP5 | LCK | |
| Nr. | Symbol | (202554_s_at) | (202779_s_at) | (203028_s_at) | (204638_at) | (204891_s_at) | ZBTB16 (205883_at) |
| 1 | COL1A2 | 0.22 | −0.69 | −0.26 | −0.30 | −0.32 | 0.38 |
| 2 | COL1A1 | 0.20 | −0.67 | −0.35 | −0.26 | −0.34 | 0.29 |
| 3 | COL3A1 | 0.08 | −0.67 | −0.32 | −0.32 | −0.33 | 0.28 |
| 4 | COL1A2 | 0.20 | −0.70 | −0.34 | −0.36 | −0.35 | 0.28 |
| 5 | SPARC | 0.26 | −0.69 | −0.34 | −0.29 | −0.34 | 0.31 |
| 6 | COL6A1 | 0.02 | −0.71 | −0.29 | −0.26 | −0.26 | 0.36 |
| 7 | COL6A2 | 0.03 | −0.67 | −0.22 | −0.13 | −0.21 | 0.23 |
| 8 | CSPG2 | 0.19 | −0.77 | −0.31 | −0.24 | −0.27 | 0.35 |
| 9 | DKFZp564I1922 | 0.19 | −0.66 | −0.20 | −0.27 | −0.23 | 0.27 |
| 10 | COL5A2 | 0.16 | −0.65 | −0.33 | −0.31 | −0.35 | 0.15 |
| 11 | FSTL1 | 0.00 | −0.72 | −0.23 | −0.33 | −0.23 | 0.24 |
| 12 | KIAA0992 | 0.17 | −0.72 | −0.45 | −0.42 | −0.42 | 0.30 |
| 13 | CSPG2 | 0.17 | −0.75 | −0.35 | −0.24 | −0.30 | 0.30 |
| 14 | PRSS11 | 0.18 | −0.73 | −0.37 | −0.31 | −0.34 | 0.31 |
| 15 | THBS2 | 0.21 | −0.70 | −0.29 | −0.29 | −0.32 | 0.26 |
| 16 | FBN1 | 0.20 | −0.70 | −0.29 | −0.24 | −0.25 | 0.24 |
| 17 | COL5A2 | 0.06 | −0.60 | −0.38 | −0.24 | −0.40 | 0.13 |
| 18 | SPARC | 0.16 | −0.68 | −0.40 | −0.36 | −0.41 | 0.25 |
| 19 | COL5A1 | 0.13 | −0.65 | −0.29 | −0.25 | −0.30 | 0.18 |
| 20 | AEBP1 | 0.10 | −0.60 | −0.18 | −0.11 | −0.24 | 0.09 |
| 21 | BGN /// | 0.08 | −0.68 | −0.29 | −0.12 | −0.30 | 0.28 |
| | SDCCAG33 | | | | | | |
| 22 | CDH11 | 0.00 | −0.67 | −0.34 | −0.25 | −0.31 | 0.11 |
| 23 | BGN | 0.12 | −0.75 | −0.31 | −0.22 | −0.30 | 0.35 |
| 24 | MGC3047 | 0.10 | −0.72 | −0.30 | −0.17 | −0.24 | 0.31 |
| 25 | ASPN | 0.16 | −0.68 | −0.40 | −0.37 | −0.47 | 0.28 |
| 26 | LRRC15 | 0.21 | −0.65 | −0.38 | −0.31 | −0.36 | 0.25 |
| 27 | COL5A1 | 0.10 | −0.53 | −0.28 | −0.26 | −0.33 | 0.00 |
| 28 | DCN | 0.17 | −0.69 | −0.25 | −0.34 | −0.28 | 0.25 |
| 29 | COL5A1 | 0.07 | −0.52 | −0.26 | −0.24 | −0.27 | 0.03 |
| 30 | DPYSL3 | 0.17 | −0.70 | −0.31 | −0.30 | −0.29 | 0.28 |
| 31 | PCOLCE | 0.09 | −0.56 | −0.14 | −0.27 | −0.21 | 0.14 |
| 32 | MRC2 | 0.16 | −0.67 | −0.28 | −0.21 | −0.24 | 0.24 |
| 33 | H2BFS | −0.03 | 0.03 | 0.00 | 0.01 | 0.11 | −0.28 |
| 34 | OLFML2B | 0.04 | −0.74 | −0.29 | −0.31 | −0.27 | 0.15 |
| 35 | UBE2S | −0.16 | 1.00 | 0.40 | 0.39 | 0.21 | −0.49 |
| 36 | EMP1 | 0.15 | −0.55 | −0.25 | −0.20 | −0.24 | 0.13 |
| 37 | FAP | 0.01 | −0.69 | −0.37 | −0.28 | −0.35 | 0.19 |
| 38 | SPON1 | 0.06 | −0.65 | −0.33 | −0.37 | −0.29 | 0.23 |
| 39 | LGALS8 | −0.22 | −0.28 | −0.33 | −0.16 | −0.07 | −0.03 |
| 40 | LOC83468 | 0.04 | −0.67 | −0.39 | −0.29 | −0.36 | 0.24 |
| 41 | COL8A2 | 0.04 | −0.63 | −0.31 | −0.29 | −0.36 | 0.11 |
| 42 | SDC2 | 0.00 | −0.57 | −0.25 | −0.23 | −0.34 | 0.20 |
| 43 | PDGFRL | 0.00 | −0.68 | −0.29 | −0.25 | −0.35 | 0.24 |
| 44 | C1QTNF3 | 0.09 | −0.62 | −0.52 | −0.41 | −0.38 | 0.14 |
| 45 | SPON1 | −0.06 | −0.57 | −0.29 | −0.34 | −0.25 | 0.19 |
| 46 | OMD | 0.01 | −0.67 | −0.33 | −0.32 | −0.33 | 0.25 |
| 47 | SPON1 | 0.05 | −0.71 | −0.18 | −0.28 | −0.15 | 0.21 |
| 48 | ZBTB16 | 0.23 | −0.49 | −0.29 | −0.41 | −0.17 | 1.00 |

| Correlate gene | | Genes covered in the examples | | | | |
|---|---|---|---|---|---|---|
| | Gene | H2BFS | LGALS8 | TRBV19 /// TRBC1 | | BGN /// SDCCAG33 |
| Nr. | Symbol | (208579_x_at) | (208933_s_at) | (210915_x_at) | OLFML2B (213125_at) | (213905_x_at) |
| 1 | COL1A2 | −0.11 | −0.13 | −0.40 | 0.83 | 0.80 |
| 2 | COL1A1 | −0.09 | −0.09 | −0.42 | 0.86 | 0.83 |
| 3 | COL3A1 | 0.13 | 0.00 | −0.34 | 0.83 | 0.65 |
| 4 | COL1A2 | −0.08 | 0.01 | −0.45 | 0.87 | 0.79 |
| 5 | SPARC | −0.17 | −0.09 | −0.41 | 0.83 | 0.83 |
| 6 | COL6A1 | −0.08 | −0.01 | −0.36 | 0.84 | 0.80 |
| 7 | COL6A2 | −0.08 | −0.04 | −0.27 | 0.80 | 0.84 |
| 8 | CSPG2 | −0.20 | 0.04 | −0.38 | 0.81 | 0.88 |
| 9 | DKFZp564I1922 | −0.25 | −0.02 | −0.40 | 0.83 | 0.79 |
| 10 | COL5A2 | −0.12 | 0.05 | −0.46 | 0.85 | 0.80 |
| 11 | FSTL1 | −0.05 | 0.08 | −0.31 | 0.89 | 0.74 |
| 12 | KIAA0992 | −0.11 | 0.12 | −0.59 | 0.85 | 0.81 |
| 13 | CSPG2 | −0.12 | 0.04 | −0.40 | 0.86 | 0.82 |
| 14 | PRSS11 | 0.05 | 0.02 | −0.41 | 0.86 | 0.77 |
| 15 | THBS2 | −0.07 | −0.04 | −0.44 | 0.84 | 0.82 |
| 16 | FBN1 | −0.03 | 0.02 | −0.38 | 0.84 | 0.79 |
| 17 | COL5A2 | 0.10 | 0.01 | −0.42 | 0.83 | 0.74 |
| 18 | SPARC | −0.06 | 0.03 | −0.47 | 0.89 | 0.75 |
| 19 | COL5A1 | −0.07 | −0.06 | −0.41 | 0.90 | 0.84 |
| 20 | AEBP1 | −0.02 | −0.01 | −0.27 | 0.74 | 0.85 |
| 21 | BGN /// | −0.20 | −0.01 | −0.39 | 0.72 | 1.00 |
| | SDCCAG33 | | | | | |
| 22 | CDH11 | 0.02 | 0.20 | −0.39 | 0.86 | 0.76 |
| 23 | BGN | −0.23 | −0.07 | −0.41 | 0.79 | 0.95 |
| 24 | MGC3047 | −0.06 | 0.01 | −0.36 | 0.79 | 0.88 |
| 25 | ASPN | −0.08 | 0.03 | −0.57 | 0.84 | 0.80 |
| 26 | LRRC15 | −0.05 | −0.07 | −0.47 | 0.84 | 0.80 |
| 27 | COL5A1 | 0.00 | −0.02 | −0.42 | 0.85 | 0.74 |
| 28 | DCN | −0.18 | 0.09 | −0.42 | 0.84 | 0.72 |
| 29 | COL5A1 | −0.04 | −0.06 | −0.35 | 0.87 | 0.74 |
| 30 | DPYSL3 | −0.09 | 0.00 | −0.45 | 0.79 | 0.84 |
| 31 | PCOLCE | 0.00 | −0.23 | −0.25 | 0.83 | 0.68 |
| 32 | MRC2 | −0.08 | −0.13 | −0.39 | 0.75 | 0.83 |
| 33 | H2BFS | 1.00 | 0.04 | 0.14 | −0.03 | −0.20 |
| 34 | OLFML2B | −0.03 | 0.09 | −0.36 | 1.00 | 0.72 |
| 35 | UBE2S | 0.03 | −0.28 | 0.35 | −0.74 | −0.68 |
| 36 | EMP1 | −0.35 | 0.31 | −0.42 | 0.58 | 0.61 |
| 37 | FAP | 0.02 | 0.10 | −0.41 | 0.85 | 0.74 |
| 38 | SPON1 | 0.00 | 0.09 | −0.40 | 0.87 | 0.67 |
| 39 | LGALS8 | 0.04 | 1.00 | −0.22 | 0.09 | −0.01 |
| 40 | LOC83468 | 0.04 | 0.11 | −0.41 | 0.84 | 0.70 |
| 41 | COL8A2 | 0.05 | 0.02 | −0.41 | 0.83 | 0.71 |
| 42 | SDC2 | 0.07 | 0.11 | −0.42 | 0.85 | 0.55 |
| 43 | PDGFRL | −0.01 | 0.02 | −0.39 | 0.85 | 0.73 |
| 44 | C1QTNF3 | 0.07 | 0.17 | −0.53 | 0.84 | 0.67 |
| 45 | SPON1 | 0.14 | 0.08 | −0.26 | 0.83 | 0.50 |
| 46 | OMD | −0.03 | 0.15 | −0.39 | 0.83 | 0.63 |
| 47 | SPON1 | −0.09 | 0.09 | −0.24 | 0.83 | 0.72 |
| 48 | ZBTB16 | −0.28 | −0.03 | −0.24 | 0.15 | 0.28 |

In the remaining branch of the classification tree, subsequent separation of the classes C and D from aggregate class CD is done using the following partial classifier:
1. The gene expression levels of one or more marker genes used in the partial classifiers are measured in a tumour sample.
2. The expression level for CYBA (Affymetrix Probe Set ID 203028_s_at) is compared against a threshold value of 1661. Samples with an expression level above this threshold are classified “C”, those below it are classified “D”.
3. Alternatively, the expression levels for ACP5 (204638_at) with a threshold of 703, for Affymetrix Probe Set ID 210915_x_at with a threshold of 812, or for LCK (204891_s_at) with a threshold of 259 can be used. In any of these genes, samples that exhibit expression values of the respective genes that are above their respective threshold value are classified as C, values below it as D.
4. Another example for such a classifier uses the expression level of GSTM3 (202554_s_at) with a threshold value of 752. Here, samples with an expression value below this threshold are classified as C, those above the threshold as D.

Affymetrix probeset ID and median expression for genes listed in Tables 1-3 are given in Table 4.

TABLE 3
Correlated genes for separation of class C<->D

| Correlate genes | | Genes covered in the examples | | | | | | |
|---|---|---|---|---|---|---|---|---|
| | Gene | AKR7A3 | EMP1 | GSTM3 | UBE2S | CYBA | ACP5 | LCK |
| Nr. | Symbol | (216381_x_at) | (201324_at) | (202554_s_at) | (202779_s_at) | (203028_s_at) | (204638_at) | (204891_s_at) |
| 1 | GSTM3 | 0.17 | 0.78 | 1.00 | −0.74 | −0.84 | −0.74 | −0.65 |
| 2 | CYBA | −0.25 | −0.62 | −0.84 | 0.62 | 1.00 | 0.74 | 0.74 |
| 3 | TRB@ | −0.46 | −0.69 | −0.71 | 0.38 | 0.72 | 0.79 | 0.93 |
| 4 | WAS | −0.39 | −0.50 | −0.63 | 0.30 | 0.60 | 0.66 | 0.83 |
| 5 | CD48 | −0.59 | −0.63 | −0.61 | 0.33 | 0.56 | 0.58 | 0.89 |
| 6 | KIAA0182 | −0.15 | −0.79 | −0.94 | 0.75 | 0.74 | 0.71 | 0.51 |
| 7 | ACP5 | −0.08 | −0.68 | −0.74 | 0.51 | 0.74 | 1.00 | 0.69 |
| 8 | LPXN | −0.46 | −0.54 | −0.68 | 0.39 | 0.70 | 0.61 | 0.85 |
| 9 | TRBV19 /// | −0.51 | −0.69 | −0.68 | 0.36 | 0.71 | 0.73 | 0.95 |
| | TRBC1 | | | | | | | |
| 10 | CD2 | −0.50 | −0.64 | −0.67 | 0.34 | 0.69 | 0.73 | 0.92 |
| 11 | IL10RA | −0.32 | −0.54 | −0.69 | 0.32 | 0.70 | 0.75 | 0.86 |
| 12 | IL2RG | −0.49 | −0.65 | −0.75 | 0.43 | 0.67 | 0.58 | 0.83 |
| 13 | TRB@ | −0.39 | −0.64 | −0.66 | 0.33 | 0.67 | 0.80 | 0.84 |
| 14 | CORO1A | −0.44 | −0.65 | −0.74 | 0.42 | 0.70 | 0.76 | 0.86 |
| 15 | CDW52 | −0.31 | −0.51 | −0.58 | 0.22 | 0.64 | 0.70 | 0.79 |
| 16 | CD3D | −0.57 | −0.64 | −0.65 | 0.36 | 0.68 | 0.71 | 0.94 |
| 17 | TRAC | −0.56 | −0.53 | −0.51 | 0.23 | 0.60 | 0.61 | 0.92 |
| 18 | TNFRSF7 | −0.42 | −0.60 | −0.73 | 0.36 | 0.78 | 0.72 | 0.90 |
| 19 | GIMAP4 | −0.22 | −0.58 | −0.67 | 0.16 | 0.64 | 0.72 | 0.79 |
| 20 | IL7R | −0.53 | −0.57 | −0.63 | 0.38 | 0.54 | 0.64 | 0.79 |
| 21 | TARP /// TRGV9 | −0.58 | −0.71 | −0.65 | 0.37 | 0.62 | 0.66 | 0.89 |
| 22 | CD3Z | −0.41 | −0.70 | −0.67 | 0.26 | 0.69 | 0.80 | 0.89 |
| 23 | LCK | −0.60 | −0.69 | −0.65 | 0.44 | 0.74 | 0.69 | 1.00 |
| 24 | IGHM | −0.49 | −0.53 | −0.55 | 0.18 | 0.56 | 0.50 | 0.88 |
| 25 | PTPN7 | −0.67 | −0.64 | −0.56 | 0.37 | 0.67 | 0.51 | 0.94 |
| 26 | LAT | −0.50 | −0.62 | −0.66 | 0.38 | 0.60 | 0.69 | 0.88 |
| 27 | ITK | −0.40 | −0.65 | −0.70 | 0.34 | 0.67 | 0.75 | 0.88 |
| 28 | TARP /// TRGV9 | −0.46 | −0.69 | −0.57 | 0.29 | 0.60 | 0.79 | 0.88 |
| 29 | RAC2 | −0.36 | −0.51 | −0.63 | 0.20 | 0.64 | 0.67 | 0.83 |
| 30 | PRKCB1 | −0.50 | −0.53 | −0.53 | 0.24 | 0.58 | 0.56 | 0.90 |
| 31 | CCR7 | −0.41 | −0.66 | −0.56 | 0.21 | 0.46 | 0.64 | 0.81 |
| 32 | LCK | −0.68 | −0.58 | −0.57 | 0.36 | 0.55 | 0.56 | 0.90 |
| 33 | IL21R | −0.42 | −0.61 | −0.77 | 0.47 | 0.71 | 0.78 | 0.85 |
| 34 | MS4A1 | −0.47 | −0.49 | −0.52 | 0.18 | 0.50 | 0.62 | 0.85 |
| 35 | NKG7 | −0.49 | −0.67 | −0.70 | 0.41 | 0.71 | 0.81 | 0.90 |
| 36 | GNLY | −0.44 | −0.77 | −0.76 | 0.41 | 0.76 | 0.71 | 0.91 |
| 37 | CD6 | −0.36 | −0.52 | −0.56 | 0.25 | 0.58 | 0.66 | 0.81 |
| 38 | PTPRCAP | −0.47 | −0.61 | −0.53 | 0.34 | 0.59 | 0.73 | 0.89 |
| 39 | GPR18 | −0.44 | −0.68 | −0.59 | 0.26 | 0.59 | 0.61 | 0.90 |
| 40 | PRKCB1 | −0.56 | −0.49 | −0.50 | 0.26 | 0.48 | 0.56 | 0.85 |
| 41 | ZAP70 | −0.50 | −0.58 | −0.62 | 0.33 | 0.62 | 0.70 | 0.92 |
| 42 | RAPGEF1 | −0.67 | −0.62 | −0.56 | 0.54 | 0.72 | 0.61 | 0.90 |
| 43 | MAP4K1 | −0.46 | −0.63 | −0.69 | 0.41 | 0.79 | 0.69 | 0.94 |
| 44 | XCL1 /// XCL2 | −0.52 | −0.77 | −0.78 | 0.58 | 0.82 | 0.76 | 0.91 |
| 45 | CD7 | −0.48 | −0.70 | −0.81 | 0.57 | 0.82 | 0.77 | 0.90 |
| 46 | CENTB1 | −0.62 | −0.61 | −0.67 | 0.42 | 0.66 | 0.51 | 0.92 |

| Correlate genes | | Genes covered in the examples | | | | | |
|---|---|---|---|---|---|---|---|
| | Gene | | H2BFS | LGALS8 | TRBV19 /// TRBC1 | OLFML2B | SPDEF |
| Nr. | Symbol | ZBTB16 (205883_at) | (208579_x_at) | (208933_s_at) | (210915_x_at) | (213125_at) | (213441_x_at) |
| 1 | GSTM3 | 0.19 | −0.10 | 0.26 | −0.68 | −0.22 | −0.10 |
| 2 | CYBA | −0.18 | −0.20 | 0.00 | 0.71 | 0.12 | −0.17 |
| 3 | TRB@ | 0.09 | 0.10 | 0.22 | 0.99 | 0.22 | −0.33 |
| 4 | WAS | 0.24 | 0.19 | 0.29 | 0.92 | 0.38 | −0.36 |
| 5 | CD48 | 0.22 | 0.16 | 0.27 | 0.94 | 0.17 | −0.48 |
| 6 | KIAA0182 | −0.25 | 0.08 | −0.39 | 0.56 | 0.13 | 0.25 |
| 7 | ACP5 | −0.23 | 0.03 | −0.17 | 0.73 | 0.31 | 0.09 |
| 8 | LPXN | 0.16 | 0.09 | 0.32 | 0.92 | 0.28 | −0.31 |
| 9 | TRBV19 /// | 0.10 | 0.09 | 0.28 | 1.00 | 0.15 | −0.37 |
| | TRBC1 | | | | | | |
| 10 | CD2 | 0.15 | 0.04 | 0.27 | 0.99 | 0.23 | −0.38 |
| 11 | IL10RA | 0.17 | 0.12 | 0.22 | 0.92 | 0.45 | −0.35 |
| 12 | IL2RG | 0.23 | 0.13 | 0.23 | 0.92 | 0.15 | −0.27 |
| 13 | TRB@ | 0.10 | 0.01 | 0.22 | 0.95 | 0.22 | −0.25 |
| 14 | CORO1A | 0.12 | 0.05 | 0.06 | 0.93 | 0.20 | −0.27 |
| 15 | CDW52 | 0.25 | −0.02 | 0.31 | 0.91 | 0.29 | −0.32 |
| 16 | CD3D | 0.09 | 0.06 | 0.25 | 0.99 | 0.13 | −0.35 |
| 17 | TRAC | 0.16 | 0.07 | 0.45 | 0.96 | 0.17 | −0.57 |
| 18 | TNFRSF7 | 0.09 | 0.06 | 0.26 | 0.96 | 0.24 | −0.40 |
| 19 | GIMAP4 | 0.33 | 0.00 | 0.22 | 0.90 | 0.40 | −031 |
| 20 | IL7R | 0.13 | 0.18 | 0.18 | 0.90 | 0.19 | −0.22 |
| 21 | TARP /// TRGV9 | 0.17 | 0.04 | 0.06 | 0.92 | 0.10 | −0.43 |
| 22 | CD3Z | 0.14 | −0.03 | 0.15 | 0.96 | 0.20 | −0.38 |
| 23 | LCK | 0.01 | 0.10 | 0.24 | 0.95 | −0.02 | −0.42 |
| 24 | IGHM | 0.31 | 0.15 | 0.32 | 0.91 | 0.15 | −0.52 |
| 25 | PTPN7 | 0.12 | −0.05 | 0.29 | 0.90 | −0.04 | −0.55 |
| 26 | LAT | 0.05 | 0.21 | 0.15 | 0.92 | 0.31 | −0.36 |
| 27 | ITK | 0.15 | 0.08 | 0.09 | 0.93 | 0.31 | −0.32 |
| 28 | TARP /// TRGV9 | 0.07 | −0.04 | 0.19 | 0.93 | 0.09 | −0.29 |
| 29 | RAC2 | 0.26 | 0.06 | 0.27 | 0.93 | 0.31 | −0.38 |
| 30 | PRKCB1 | 0.20 | 0.16 | 0.44 | 0.92 | 0.26 | −0.61 |
| 31 | CCR7 | 0.19 | 0.18 | 0.15 | 0.90 | 0.12 | −0.25 |
| 32 | LCK | −0.03 | 0.24 | 0.25 | 0.91 | −0.08 | −0.33 |
| 33 | IL21R | 0.03 | 0.11 | 0.13 | 0.92 | 0.31 | −0.15 |
| 34 | MS4A1 | 0.18 | 0.22 | 0.36 | 0.93 | 0.24 | −0.41 |
| 35 | NKG7 | −0.16 | 0.04 | 0.10 | 0.92 | 0.05 | −0.21 |
| 36 | GNLY | 0.14 | −0.04 | 0.07 | 0.91 | −0.05 | −0.23 |
| 37 | CD6 | 0.00 | 0.11 | 0.42 | 0.90 | 0.42 | −0.40 |
| 38 | PTPRCAP | 0.06 | 0.06 | 0.35 | 0.92 | 0.18 | −0.37 |
| 39 | GPR18 | 0.23 | 0.22 | 0.23 | 0.90 | 0.18 | −0.53 |
| 40 | PRKCB1 | 0.16 | 0.22 | 0.44 | 0.93 | 0.20 | −0.39 |
| 41 | ZAP70 | −0.04 | 0.16 | 0.14 | 0.88 | 0.11 | −0.31 |
| 42 | RAPGEF1 | −0.26 | 0.06 | 0.29 | 0.81 | −0.23 | −0.30 |
| 43 | MAP4K1 | 0.11 | 0.06 | 0.19 | 0.93 | 0.18 | −0.49 |
| 44 | XCL1 /// XCL2 | −0.07 | 0.11 | 0.04 | 0.90 | 0.01 | −0.25 |
| 45 | CD7 | −0.08 | 0.07 | 0.03 | 0.87 | 0.14 | −0.23 |
| 46 | CENTB1 | 0.11 | 0.16 | 0.24 | 0.90 | 0.09 | −0.46 |

| Correlate genes | | Genes covered in the examples | | | |
|---|---|---|---|---|---|
| Nr. | Gene Symbol | BGN /// SDCCAG33 (213905_x_at) | SPDEF (214404_x_at) | MLPH (218211_s_at) | SPDEF (220192_x_at) |
| 1 | GSTM3 | 0.13 | −0.38 | 0.29 | −0.21 |
| 2 | CYBA | −0.35 | 0.06 | −0.21 | −0.21 |
| 3 | TRB@ | −0.04 | 0.16 | −0.19 | −0.08 |
| 4 | WAS | 0.07 | 0.10 | −0.24 | −0.05 |
| 5 | CD48 | −0.02 | 0.10 | −0.16 | −0.12 |
| 6 | KIAA0182 | −0.15 | 0.45 | −0.34 | 0.31 |
| 7 | ACP5 | 0.06 | 0.17 | −0.69 | 0.04 |
| 8 | LPXN | −0.08 | 0.20 | −0.11 | −0.14 |
| 9 | TRBV19 /// | −0.10 | 0.18 | −0.09 | −0.12 |
| | TRBC1 | | | | |
| 10 | CD2 | −0.02 | 0.15 | −0.16 | −0.09 |
| 11 | IL10RA | 0.13 | 0.05 | −0.25 | −0.12 |
| 12 | IL2RG | −0.15 | 0.29 | −0.16 | 0.04 |
| 13 | TRB@ | −0.06 | 0.16 | −0.27 | −0.05 |
| 14 | CORO1A | −0.10 | 0.15 | −0.31 | −0.03 |
| 15 | CDW52 | −0.02 | 0.05 | −0.22 | −0.15 |
| 16 | CD3D | −0.12 | 0.23 | −0.07 | −0.11 |
| 17 | TRAC | −0.04 | 0.03 | −0.04 | −0.20 |
| 18 | TNFRSF7 | −0.13 | 0.13 | −0.14 | −0.06 |
| 19 | GIMAP4 | 0.16 | 0.02 | −0.30 | −0.03 |
| 20 | IL7R | −0.08 | 0.31 | −0.22 | 0.06 |
| 21 | TARP /// TRGV9 | 0.03 | 0.19 | 0.01 | −0.03 |
| 22 | CD3Z | 0.02 | 0.05 | −0.21 | −0.10 |
| 23 | LCK | −0.24 | 0.13 | −0.01 | −0.28 |
| 24 | IGHM | −0.06 | 0.03 | 0.00 | −0.23 |
| 25 | PTPN7 | −0.19 | 0.06 | 0.11 | −0.38 |
| 26 | LAT | 0.09 | 0.15 | −0.18 | −0.11 |
| 27 | ITK | 0.07 | 0.06 | −0.28 | −0.16 |
| 28 | TARP /// TRGV9 | 0.01 | 0.17 | −0.09 | −0.09 |
| 29 | RAC2 | −0.03 | 0.03 | −0.26 | −0.12 |
| 30 | PRKCB1 | 0.05 | −0.03 | −0.09 | −0.21 |
| 31 | CCR7 | 0.03 | 0.26 | −0.04 | 0.01 |
| 32 | LCK | −0.32 | 0.37 | 0.06 | −0.02 |
| 33 | IL21R | −0.02 | 0.28 | −0.26 | 0.04 |
| 34 | MS4A1 | −0.02 | 0.10 | −0.21 | −0.06 |
| 35 | NKG7 | −0.19 | 0.30 | −0.06 | 0.03 |
| 36 | GNLY | −0.23 | 0.19 | −0.09 | −0.12 |
| 37 | CD6 | 0.14 | 0.10 | −0.09 | −0.15 |
| 38 | PTPRCAP | 0.02 | 0.04 | −0.18 | −0.23 |
| 39 | GPR18 | 0.05 | −0.04 | −0.12 | −0.23 |
| 40 | PRKCB1 | −0.03 | 0.23 | −0.06 | −0.06 |
| 41 | ZAP70 | −0.11 | 0.13 | −0.12 | −0.14 |
| 42 | RAPGEF1 | −0.49 | 0.23 | 0.06 | −0.28 |
| 43 | MAP4K1 | −0.10 | 0.02 | −0.05 | −0.22 |
| 44 | XCL1 /// XCL2 | −0.23 | 0.30 | −0.02 | −0.05 |
| 45 | CD7 | −0.20 | 0.13 | −0.32 | −0.16 |
| 46 | CENTB1 | −0.19 | 0.14 | −0.05 | −0.21 |

TABLE 4
Correlated genes, Probeset ID and median expression

| | Affymetrix |
|---|---|
| Gene Symbol | Probeset ID |
| ACP5 | 204638_at |
| AEBP1 | 201792_at |
| AGR2 | 209173_at |
| AKR7A3 | 206469_x_at |
| AKR7A3 | 216381_x_at |
| ASPN | 219087_at |
| BGN | 201261_x_at |
| BGN /// SDCCAG33 | 213905_x_at |
| C1QTNF3 | 220988_s_at |
| C6orf29 | 205597_at |
| CCR7 | 206337_at |
| CD2 | 205831_at |
| CD3D | 213539_at |
| CD3Z | 210031_at |
| CD48 | 204118_at |
| CD6 | 211893_x_at |
| CD7 | 214551_s_at |
| CDH11 | 207173_x_at |
| CDW52 | 204661_at |
| CENTB1 | 205213_at |
| COL1A1 | 202310_s_at |
| COL1A2 | 202403_s_at |
| COL1A2 | 202404_s_at |
| COL3A1 | 201852_x_at |
| COL5A1 | 203325_s_at |
| COL5A1 | 212488_at |
| COL5A1 | 212489_at |
| COL5A2 | 221729_at |
| COL5A2 | 221730_at |
| COL6A1 | 213428_s_at |
| COL6A2 | 209156_s_at |
| COL8A2 | 221900_at |
| CORO1A | 209083_at |
| CSPG2 | 204620_s_at |
| CSPG2 | 221731_x_at |
| CYBA | 203028_s_at |
| DCN | 209335_at |
| DKFZp564I1922 | 209596_at |
| DPYSL3 | 201431_s_at |
| EMP1 | 201324_at |
| FAP | 209955_s_at |
| FBN1 | 202766_s_at |
| FOXA1 | 204667_at |
| FOXC1 | 213260_at |
| FSTL1 | 208782_at |
| GATA3 | 209602_s_at |
| GATA3 | 209603_at |
| GATA3 | 209604_s_at |
| GIMAP4 | 219243_at |
| GNLY | 205495_s_at |
| GPR18 | 210279_at |
| GSTM3 | 202554_s_at |
| H2BFS | 208579_x_at |
| IGHM | 212827_at |
| IL10RA | 204912_at |
| IL21R | 221658_s_at |
| IL2RG | 204116_at |
| IL7R | 205798_at |
| ITK | 211339_s_at |
| KIAA0182 | 212057_at |
| KIAA0992 | 200897_s_at |
| LAT | 211005_at |
| LCK | 204890_s_at |
| LCK | 204891_s_at |
| LGALS8 | 208933_s_at |
| LOC400451 | 51158_at |
| LOC83468 | 221447_s_at |
| LPXN | 216250_s_at |
| LRRC15 | 213909_at |
| MAP4K1 | 206296_x_at |
| MGC3047 | 213422_s_at |
| MLPH | 218211_s_at |
| MRC2 | 37408_at |
| MS4A1 | 217418_x_at |
| MYB | 204798_at |
| NKG7 | 213915_at |
| OLFML2B | 213125_at |
| OMD | 205907_s_at |
| PCOLCE | 202465_at |
| PDGFRL | 205226_at |
| PH-4 | 222125_s_at |
| PRKCB1 | 207957_s_at |
| PRKCB1 | 209685_s_at |
| PRSS11 | 201185_at |
| PTPN7 | 204852_s_at |
| PTPRCAP | 204960_at |
| RAC2 | 207419_s_at |
| RAPGEF1 | 204543_at |
| SDC2 | 212157_at |
| SPARC | 200665_s_at |
| SPARC | 212667_at |
| SPDEF | 213441_x_at |
| SPDEF | 214404_x_at |
| SPDEF | 220192_x_at |
| SPON1 | 209436_at |
| SPON1 | 209437_s_at |
| SPON1 | 213994_s_at |
| TARP /// TRGV9 | 209813_x_at |
| TARP /// TRGV9 | 215806_x_at |
| THBS2 | 203083_at |
| TNFRSF7 | 206150_at |
| TRAC | 209670_at |
| TRB@ | 211796_s_at |
| TRB@ | 213193_x_at |
| TRBV19 /// TRBC1 | 210915_x_at |
| UBE2S | 202779_s_at |
| VAV3 | 218807_at |
| WAS | 38964_r_at |
| XCL1 /// XCL2 | 214567_s_at |
| ZAP70 | 214032_at |
| ZBTB16 | 205883_at |

### Example 3

### Significance of Correlated Marker Genes (A Theoretical Example)

It is well known that expression level data of multiple genes can be highly redundant information, due to co-regulation of certain genes or groups of genes in living organisms.

According to the invention, the so-called “correlation coefficient” is used as a measure for the degree of similarity of expression levels in multiple samples. If we denote the log expression value of the i-th gene (i=1, 2, 3, . . . N) of patient j (j=1, 2, 3, . . . M) by g_{i,j}, the correlation coefficient r may be defined as

$${r}_{i 1 , i 2} := \frac{{\sum }_{j = 1}^{M} ( {g}_{i 1 , j} - {\overline{g}}_{i 1} ) \cdot ( {g}_{i 2 , j} - {\overline{g}}_{i 2} )}{\sqrt{( {\sum }_{j = 1}^{M} {( {g}_{i 1 , j} - {\overline{g}}_{i 1} )}^{2} ) \cdot ( {\sum }_{j = 1}^{M} {( {g}_{i 2 , j} - {\overline{g}}_{i 2} )}^{2} )}}$$

where the mean value of gene i is given by

$${\overline{g}}_{i} := \frac{1}{M} {\sum }_{j = 1}^{M} {g}_{i , j} .$$

r is also called “Pearson Correlation Coefficient” and is widely used in the statistical community.

While r may take any value between (and including) −1 and 1, correlations with an absolute value close to 1 indicate a linear relationship between the genes under consideration, meaning that the two genes carry virtually the same information.

In the context of the present invention it is apparent that genes sharing a sufficiently large correlation coefficient with marker genes of the preceding examples can equally well be used in the classification method, because they provide almost identical information.

Tables 1-3 list genes with a high correlation to marker genes to marker genes used in the Examples. They can be used in the separation of breast cancer response classes AB and CD from ABCD (Table 1), and for the separation of breast cancer response classes A and B from AB (Table 2), and finally for the separation of breast cancer response classes C and D (Table 3) from CD.

A “sufficiently large correlation coefficient”, in this context, needs to be explained in more detail. To keep the gene lists fair and short, we identified genes that had an unusually high correlation with a probability of p<0.05 already including a conservative Bonferroni correction (that is, p has to be divided by the number of genes checked for high correlation, in this case, N=22284 for Affymetrix HG U133A chip used here) which yielded an effective p value of p_{eff}<0.05/22284=2.24e−6.

Using a (two-sided) Student's t statistic, we can compute the minimum correlation coefficient rₘᵢₙ from p_{eff}, also taking the sample number at each separation point into account.

Finally, the following minimal correlation values and numbers of correlated genes were obtained:

| | Number of samples | | Resulting number |
|---|---|---|---|
| Separation | in finding cohort | rₘᵢₙ | of correlated genes |
| AB <-> CD | 57 | 0.8160 | 17 |
| A <-> B | 42 | 0.8270 | 48 |
| C <-> D | 15 | 0.9013 | 46 |

Thus, genes having a correlation coefficient equal to or larger than rₘᵢₙ to the marker genes of Example 2 of the present invention, are further preferred marker genes for the separation of AB and CD, A and B, and C and D in a classification tree of the invention.

Further preferred marker genes are genes whose gene expression is correlated with the one of marker genes of Example 2 with a correlation coefficient in one of Tables 1, 2 or 3 of preferably 0.7, 0.9, 0.95, 0.99, 0.999 or most preferably 1.

Also preferred marker genes are genes whose gene expression is correlated with at least one marker gene of Example 2 with a correlation coefficient of preferably 0.7, 0.9, 0.95, 0.99 or most preferably 1 in a separate series of expression level measurements.

Further preferred marker genes are genes whose gene expression is previously known to be highly correlated with one of marker genes of Example 2.

### Example 4

### Advantage of Bivariate Classification Over Univariate Classification in Certain Cases

The bivariate classification is in many cases superior to previously used univariate models because it succeeds in situations where the latter fail. This can be illustrated by considering the following (theoretical) example:

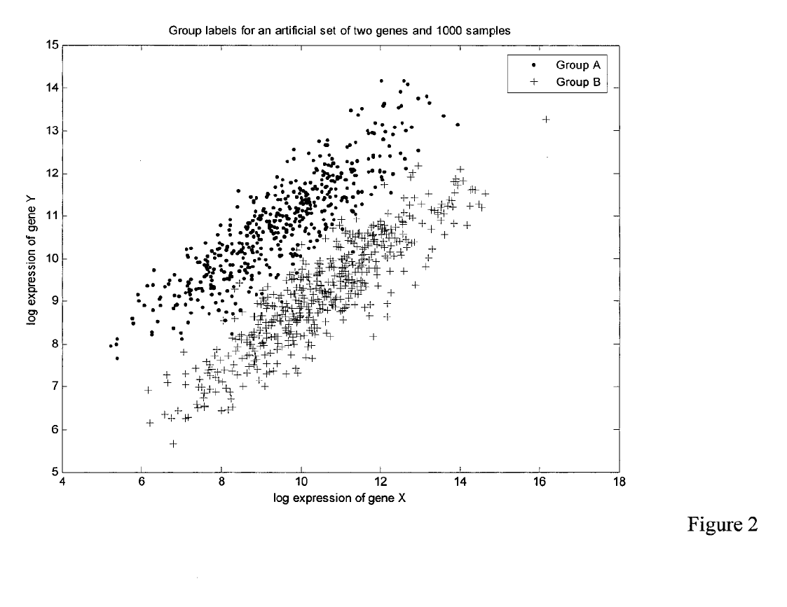

An artificial data set is assumed. This dataset contains expression level measurements of two genes (Gene X and Gene Y) in two groups of samples (classes A and B). Each group consists of 500 samples. The data is shown in FIG. 2.

The task is to find a mathematical classification operator, i.e., an algorithm that predicts to which class a given sample with measured gene expression g1 of Gene X and g2 of Gene Y belongs.

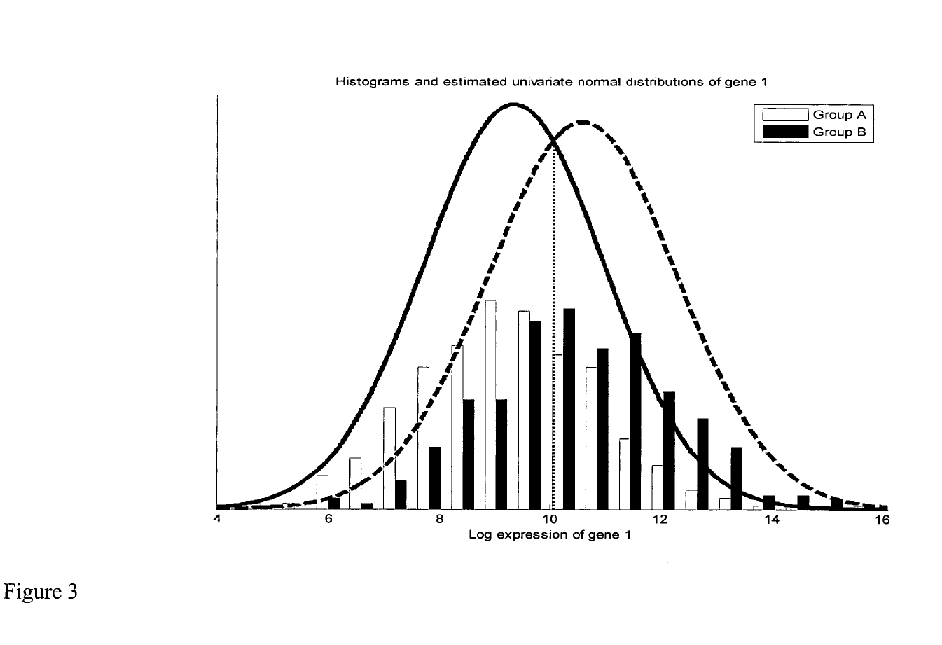

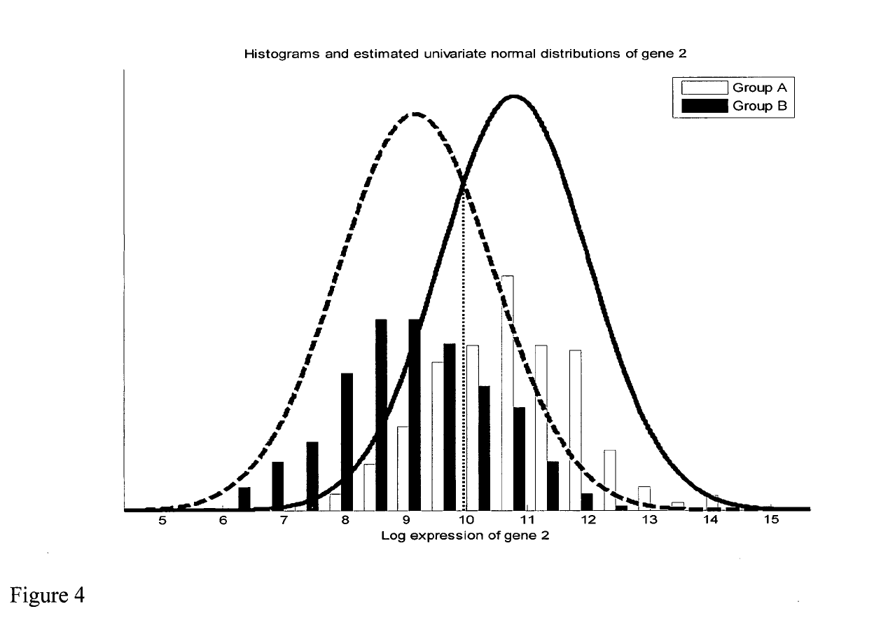

The simplest approach is to take a univariate approach, that is, to build an algorithm on the expression of just one gene. One such model is to approximate the histograms of the data by two normal distributions, one for each group. The two parameters for each normal distribution, mean value and standard deviation, can be estimated from the data. Results of this model are graphically represented in FIG. 3 for Gene X, and in FIG. 4 for Gene Y.

For an unknown sample, one computes the probabilities for each of the groups on basis of the normal distributions, and the more likely group is chosen as the predicted group. This is roughly the same as the definition of a threshold value between the mean values of the two distributions.

The result for a classification operator based on Gene X only is a threshold value of about 10.016 with the rule
If the expression of Gene X is less than or equal to 10.016, then the sample is in group 1, otherwise it is in group 2.

The results for the classification is as follows

| | Predicted A | Predicted B |
|---|---|---|
| Is A | 325 | 175 |
| Is B | 175 | 325 |

which accounts for an overall correctness of 65%.

On the other hand, a univariate classification operator solely based on the expression of Gene Y yields a threshold value of 10.013 and the following results:

| | Predicted A | Predicted B |
|---|---|---|
| Is A | 377 | 123 |
| Is B | 128 | 372 |

The overall correctness is now 74.9%.

Both overall correctness values account for poor prediction quality even on the training set. A random assignment of data to one of the classes has an expected overall correctness of 50%, and both 65% and 74.9% cannot be considered satisfactory in this context.

A bivariate separation strategy makes formally the same assumption about the structure of the data:

Each group can be modelled as a normal distribution, only this time both genes are used at the same time (hence this separation strategy is termed “bivariate”). Again, the parameters (mean value μ and covariance matrix Σ², the latter of which takes the place of the variance σ² in the univariate case) can be estimated from the data.

As in the univariate case, a classification algorithm evaluated the in-class-probabilities for an unknown sample based on its expression of both genes. The classifier then chooses the more likely class.

For the data at hand, the estimated parameters of the bivariate normal distribution for the first group is

$${μ}_{1} = ( \begin{matrix}9.45 \\ 10.86\end{matrix} ) , {Σ}_{1}^{2} = ( \begin{matrix}2.68 & 1.78 \\ 1.78 & 1.50\end{matrix} ) ,$$

and for the second group

$${μ}_{2} = ( \begin{matrix}10.59 \\ 9.17\end{matrix} ) , {Σ}_{2}^{2} = ( \begin{matrix}2.67 & 1.72 \\ 1.72 & 1.50\end{matrix} ) .$$

On the training data, the following classification is produced:

| | Predicted A | Predicted B |
|---|---|---|
| Is A | 491 | 9 |
| Is B | 11 | 489 |

This corresponds to an overall correctness of 98%, which clearly outperforms both of the univariate classification rules. Thus, classification of breast cancer tumours is advantageously based on bivariate classification, in certain cases.

### Example 5

### Bivariate Classification of Tumour Samples

As previously defined, a classification maps gene expression levels obtained in the analysis of a given tumor tissue sample to one of two or more predefined groups. In this example, details about the derivation of a bivariate classification will be given for the special but important case of a bivariate binary classification, i.e. the classification of a tumor sample into one of two classes (aggregate or elementary) based on the expression levels of two genes (simultaneously) in a tumor sample.

As a preliminary, a set of tumor tissue sample is given in advance to obtain an optimal combination of genes along with an optimal set of parameters. This step is called the “training” of the classification operator. It will be assumed that there are classes A and B with NA (resp. NB) different tumor samples, and that NA and NB are sufficiently large. Let N=NA+NB denote the total number of samples in the training set. For each of the tumor samples (regardless of its class), M gene expression levels are given. Let gij (with i=1, 2, . . . , N and j=1, 2, . . . , M) denote the (log) expression of gene j in sample i. The logarithm may be taken to any base >1, but in the context of the invention at hand a choice of base-2 logarithms has been made. Finally, let cl_i (with i=1, 2, . . . , N) be the class of sample i, where cl_i=0 means that sample i belongs to class A, and cl_i=1 if it belongs to class B.

The assumption made in the approach is that the samples in each group (A or B) are random samples drawn from a group-inherent bivariate Gaussian distribution with group-wise mean vector mu_group and group-wise correlation matrix Sigma_group (group=A, B). The objective is to make an optimal choice a) for the genes used in the distributions, and b) to propose optimal values for the mean vectors and the covariance matrices.

Since the search for the optimal gene pair is done exhaustively, it is very favourable in terms of computational effort and statistical significance to restrict the discovery to a small set of genes only. The idea here is to avoid the use of non-informative, low-expression, highly noisy genes. This can be achieved using an (unsupervised) filter.

For each pair of genes (r,s), a cross-validation procedure is implemented by separating the entire training set randomly into two sets, “Set 1” (containing 80% of the samples of each group), and “Set 2” (containing the remaining samples of each group). For Set 1, μ_{A} and Σ_{A} are then estimated by the following formulae obvious to a person skilled in the art:

$${μ}_{A} := \frac{1}{0.8 \cdot {N}_{A}} \cdot {\sum }_{i ∈ Set 1 , cl ( i ) = 0}^{ } ( \begin{matrix}{g}_{is} \\ {g}_{it}\end{matrix} ) , {Σ}_{A}^{2} := \frac{1}{0.8 \cdot {N}_{A} - 1} \cdot {\sum }_{i ∈ Set 1 , cl ( i ) = 0} {( ( \begin{matrix}{g}_{is} \\ {g}_{it}\end{matrix} ) - {μ}_{A} )}^{′} ( ( \begin{matrix}{g}_{is} \\ {g}_{it}\end{matrix} ) - {μ}_{A} )$$

$${μ}_{B} := \frac{1}{0.8 \cdot {N}_{B}} \cdot {\sum }_{i ∈ Set 1 , cl ( i ) = 1}^{ } ( \begin{matrix}{g}_{is} \\ {g}_{it}\end{matrix} ) , {Σ}_{B}^{2} := \frac{1}{0.8 \cdot {N}_{B} - 1} \cdot {\sum }_{i ∈ Set 1 , cl ( i ) = 1} {( ( \begin{matrix}{g}_{is} \\ {g}_{it}\end{matrix} ) - {μ}_{B} )}^{′} ( ( \begin{matrix}{g}_{is} \\ {g}_{it}\end{matrix} ) - {μ}_{B} )$$

These values, obtained solely on Set1, are then used to assess the quality of this candidate predictor; for each sample k in Set2, the probabilities

$${pr}_{k , A} := \frac{1}{2 π \sqrt{det {Σ}_{A}^{2}}} \cdot exp ( - \frac{1}{2} \cdot {( ( \begin{matrix}{g}_{ks} \\ {g}_{kt}\end{matrix} ) - {μ}_{A} )}^{′} {Σ}_{A}^{- 2} ( ( \begin{matrix}{g}_{ks} \\ {g}_{kt}\end{matrix} ) - {μ}_{A} ) )$$

$${pr}_{k , B} := \frac{1}{2 π \sqrt{det {Σ}_{B}^{2}}} \cdot exp ( - \frac{1}{2} \cdot {( ( \begin{matrix}{g}_{ks} \\ {g}_{kt}\end{matrix} ) - {μ}_{B} )}^{′} {Σ}_{A}^{- 2} ( ( \begin{matrix}{g}_{ks} \\ {g}_{kt}\end{matrix} ) - {μ}_{B} ) )$$

are computed. pr_{k,A} is the in-class probability of sample k for class A, and pr_{k,B} the in-class probability of sample k for class B, respectively.

From these two quantities, a class is predicted:

$$pred ( k ) := \{ \begin{matrix}A & {pr}_{k , A} > {pr}_{kB} \\ B & {pr}_{k , A} < {pr}_{k , B}\end{matrix}$$

From the predicted class and the known class, an overall correctness is computed:

$$oc = \frac{{\sum }_{k ∈ set 2 , pred ( k ) = cl ( k )} 1}{{\sum }_{k ∈ set 2 }^{ } 1} .$$

The cross validation is carried out many times (e.g. 100 times) while the overall correctness is averaged over all cross validations. Finally, the gene pair with the best (largest) average overall correctness is chosen, and the mean values μ_{A}, μ_{B} and the covariance matrices Σ_{A}, Σ_{B} are re-computed using the entire training set.

### Example 6

### Determination of Thresholds for Univariate Classification

In the univariate case, an analogous approach compared to the bivariate classification described in Example 5 was chosen. While the bivariate parameter estimation made the assumption of a (bivariate) normal distribution, the assumption for the univariate case consequently is a univariate normal distribution.

With the preliminaries (training set, gene expression values, group assignments) as in the bivariate case, the objective for the univariate case was to obtain an optimal single gene with an optimal threshold value for class prediction.

Again, the search throughout the genes was exhaustive. For each chosen gene k, a separation into set “Set 1” and “Set 2” was done using the same approach as in the bivariate case.

For given sets, univariate Gaussian distributions were estimated from Set 1, namely:

$${μ}_{A} := \frac{1}{0.8 \cdot {N}_{A}} \cdot {\sum }_{i ∈ Set 1 , cl ( i ) = 0}^{ } {g}_{ik} , {σ}_{A}^{2} := \frac{1}{0.8 \cdot {N}_{A} - 1} \cdot {\sum }_{i ∈ Set 1 , cl ( i ) = 0} {( {g}_{is} - {μ}_{A} )}^{2}$$

$${μ}_{B} := \frac{1}{0.8 \cdot {N}_{B}} \cdot {\sum }_{i ∈ Set 1 , cl ( i ) = 1}^{ } {g}_{ik} , {σ}_{B}^{2} := \frac{1}{0.8 \cdot {N}_{B} - 1} \cdot {\sum }_{i ∈ Set 1 , cl ( i ) = 1} {( {g}_{is} - {μ}_{B} )}^{2}$$

Here, the scalar parameter σ_{A} (or rather, its squared value σ_{A}², the variance in class A) takes the place of the covariance matrix Σ_{A}² of the bivariate case.

The estimated distribution functions were used to compute predicted classes for all samples k in Set 2:

$${pr}_{k , A} := \frac{1}{\sqrt{2 π \cdot σ}} \cdot exp ( - \frac{{( {g}_{ks} - {μ}_{A} )}^{2}}{2 {σ}_{A}^{2}} )$$

$${pr}_{k , B} := \frac{1}{\sqrt{2 π \cdot {σ}_{B}}} \cdot exp ( - \frac{{( {g}_{ks} - {μ}_{B} )}^{2}}{2 {σ}_{B}^{2}} )$$

Proceeding as in the bivariate case, classes were predicted for all samples in Set 2, and an overall correctness could be computed that was then averaged over all cross validations. The highest average overall correctness then determined the best gene for the univariate separation, and the four parameters μ_{A}, σ_{A}, μ_{B}, and σ_{B} were re-computed to get a more reliable estimate.

As a remark, the prediction operator can be greatly simplified to a simple threshold value in most cases by inserting the definition of pr_{k,A} and pr_{k,B} for each sample and computing the values of gₖₛ where the two probabilities coincide. The details of this computation is straight-forward and very obvious for a person skilled in the art, so we spare any details here.

### CITED LITERATURE

(1) Chang J C, Wooten E C, Tsimelzon A, Hilsenbeck S G, Gutierrez M C, Elledge R, Mohsin S, Osborne C K, Chamness G C, Allred D C, O'Connell P. Gene expression profiling for the prediction of therapeutic response to docetaxel in patients with breast cancer. Lancet, 362:362-369, 2003.
(2) Goldhirsch A, Wood W C, Gelber R D, Coates A S, Thulimann B, Senn H J. Meeting Highlights: updated international expert consensus on the primary therapy of early breast cancer. J Clin Oncol 21: 3357-3365, 2003
(3) Early Breast Cancer Trialists' Collaborative Group. Polychemotherapy for early breast cancer: an overview of the randomised trials. Lancet 352: 930-942, 1998
(4) Early Breast Cancer Trialists' Collaborative Group. Tamoxifen for early breast cancer: an overview of the randomised trials. Lancet 351: 1451-1467, 1998
(5) Ganz P A, Desmond K A, Leedham B, Rowland J H, Meyerowitz B E, Belin T R. Quality of life in long-term, disease-free survivors of breast cancer: a follow-up study. J Natl Cancer Inst 94: 39-49, 2002
(6) Ayers M, Symmans W F, Stec J, Damokosh A I, Clark E, Hess K, Lecocke M, Metivier J, Booser D, Ibrahim N, Valero V, Royce M, Arun B, Whitman G, Ross J, Sneige N, Hortobagyi G N, Pusztai L. Gene expression profiles predict complete pathologic response to neoadjuvant paclitaxel and fluorouracil, doxorubicin, and cyclophosphamide chemotherapy in breast cancer. J Clin Oncol 22(12): 2284-22932004
(7) Hannemann J, Oosterkamp H M, Bosch C A, Velds A, Wessels L F, Loo C, Rutgers E J, Rodenhuis S, van de Vijver M J. Changes in gene expression associated with response to neoadjuvant chemotherapy in breast cancer. J Clin Oncol. 2005 23(15):333142, 2005
(8). Rouzier R, et al. Breast cancer molecular subtypes respond differently to preoperative chemotherapy. Clin Cancer Res 11: 5678-85, 2005
(9) Van't Veer L J, Dai H, van de Vijver M J, He Y D, Hart A A M, Mao M, Peterse H L, van der Kooy K, Marton M J, Witteveen A T, Schreiber G J, Kerkhoven R M, Roberts C, Linsley P S, Bernards R, Friend S H. Gene expression profiling predicts clinical outcome of breast cancer. Nature 415: 530-536, 2002
(10) Wang Y, Klijn J G M, Zhang Y, Sieuwerts A M, Look M P, Yang F, Talantov D, Timmermans M, Meijer-van Gelder M E, Yu J, Jatkoe T, Berns E M J J, Atkins D, Foekens J A. Lancet 365: 671-679, 2005
(11) Paik S, Shak S, Tang G, Kim C, Baker J, Cronin M, Baehner F L, Walker M G, Watson D, Park T, Hiller W, Fisher E R, Wickerham D L, Bryant J, Wolmark N. A multigene assay to predict recurrence of tamoxifen-treated, node-negative breast cancer. N Engl J Med

1. Method for the prediction of the response to epirubicin/cyclophosphamide-based chemotherapy of a breast cancer in a patient, from a tumour sample of said patient, comprising steps of
(a) determining the expression level of a group of marker genes consisting of
(i) a first marker gene selected from the group consisting of MLPH, SPDEF, and AKR7A3; and
(ii) a pair of second marker genes selected from the group of pairs consisting of (H2BFS and UBE2S), (BGN and ZBTB16), (ZBTB16 and EMP1), (LGALS8 and UBE2S) and (OLFML2B and ZBTB16); and
(iii) a third marker gene selected from the group consisting of CYBA, ACP5, a gene specifically binding to Affymetrix probe set ID 210915_xat, LCK, GSTM3;
(b) classifying said sample as belonging to one of several breast cancer response classes from the expression levels determined under (a);
(c) predicting the response of said breast cancer in said patient to chemotherapy from previously known characteristic properties of tumours of said one of several breast cancer response classes.

2. Method of claim 1, wherein said several breast cancer response classes are four breast cancer response classes.

3. Method of claim 1, wherein at least one marker gene of said group of marker genes is substituted by a substitute marker gene, said substitute marker gene being coregulated with said at least one marker gene.

4. Method of claim 3, wherein said substitute marker gene has an absolute correlation coefficient to said at least one marker gene of equal to or higher than
(a) 0.816 in Table 1, if said marker gene is MLPH, SPDEF or AXR7A3;
(b) 0.827 in Table 2, if said marker gene is H2BFS, UBE2S, BGN, ZBTB16, EMP1, LGALS8 or OLFML2B; and
(c) 0.9013 in Table 3, if said marker gene is CYBA, ACP5, a gene specifically binding to Affymetrix probe set ID 210915_x-at, LCK or GSTM3.

5. Method of claim 3, wherein said classification step (b) is based on a mathematical discriminant function.

6. Method of claim 3, wherein said classification in step (b) is based on a decision tree.

7. Method of claim 6, wherein said decision tree involves at least one bivariate classification step.

8. Method of claim 1, wherein said classification uses a k-nearest-neighbour (kNN) algorithm.

9. Method of claim 1, wherein the chemotherapy is a neoadjuvant chemotherapy.

10. Method of claim 1, wherein the response to chemotherapy is clinical response or pathological response.

11. Method of claim 1, wherein said patient is a human patient.

12. Method of claim 1, wherein said sample of a tumour is a fixed sample, a paraffin-embedded sample, a fresh sample, a fresh frozen sample or a frozen sample.

13. Method of claim 1, wherein said sample of a tumour is from fine needle biopsy, core biopsy or fine needle aspiration.

14. Method of claim 1, wherein said determination of the expression level is by microarray experiment, by RT-PCR, by SAGE, by immunohistochemistry or by TaqMan.

15. A microarray comprising immobilized nucleic acid probes capable of specific hybridization with
a) a first marker gene selected from the group consisting of MLPH, SPDEF, and AKR7A3; and
b) two second marker genes in a pair selected from the group of pairs consisting of (H2BFS and UBE2S), (BGN and ZBTB16), (ZBTB16 and EMP1), (LGALS8 and UBE2S) and (OLFML2B and ZBTB16); and with
c) a third marker gene selected from the group consisting of CYBA, ACP5, a gene specifically binding to Affymetrix probe set ID 21091 5_x_at, LCK, GSTM3.

16. A microarray of claim 16, wherein said microarray is an RNA array or a DNA array.

17. A system for predicting the response of a breast cancer in a patient to chemotherapy, comprising
a) means for determining the expression level of a group of marker genes consisting of
i) a first marker gene selected from the group consisting of MLPH, SPDEF, and AKR7A3; and
ii) a pair of second marker genes selected from the group of pairs consisting of (H2BFS and UBE2S), (BGN and ZBTB16), (ZBTB16 and EMP1), (LGALS8 and UBE2S) and (OLFML2B and ZBTB16); and
iii) a third marker gene selected from the group consisting of CYBA, ACP5, a gene specifically binding to Affymetrix probe set ID 210915_x_at, LCK, GSTM3.
b) computing means adapted for classifying said sample to one of several breast cancer response classes from expression levels of said group of marker genes,
c) computing means adapted for predicting the response of said breast cancer in said patient to chemotherapy from characteristic properties of tumours of said one of several breast cancer response class.

18. A system of claim 17, wherein said several breast cancer response classes are four (4) breast cancer response classes.

19. System of claim 18, wherein said means for determining the expression level of a group of marker genes comprises a microarray, a system for 2D gel electrophoresis, a SAGE system or a system for immunohistochemical determination of expression levels.
